SEQUENCE LISTING (10 sequences)

SEQ ID NO: 1 - DNA, 12 bp, Artificial sequence
  Synthetic oligonucleotide
ccgcaacagtaa

SEQ ID NO: 2 - DNA, 12 bp, Artificial sequence
  Synthetic oligonucleotide
ttactgttgcgg

SEQ ID NO: 3 - DNA, 13 bp, Artificial sequence
  Synthetic oligonucleotide
tnnncgcancant

SEQ ID NO: 4 - DNA, 24 bp, Artificial sequence
  Synthetic oligonucleotide
ggactacttcgcgcaacagtataa

SEQ ID NO: 5 - DNA, 24 bp, Artificial sequence
  Synthetic oligonucleotide
tttcagctttccgcaacagtaaaa

SEQ ID NO: 6 - DNA, 18 bp, Artificial sequence
  Synthetic oligonucleotide
tagggataacagggtaat

SEQ ID NO: 7 - DNA, 18 bp, Artificial sequence
  Synthetic oligonucleotide
attaccctgttatcccta

SEQ ID NO: 8 - DNA, 25 bp, Artificial sequence
  Synthetic oligonucleotide
gccttgccgggtaagttccggcgcg

SEQ ID NO: 9 - DNA, 24 bp, Artificial sequence
  Synthetic oligonucleotide
cgcgccggaacttaccggcaaggc

SEQ ID NO: 10 - DNA, 24 bp, Artificial sequence
  Synthetic oligonucleotide
tttcagctttccgcaacagtataa

(19) United States
(12) Patent Application Publication
(10) Pub. No.: US 2012/0202251 A1
(43) Pub. Date: Aug. 9, 2012

(54) IN VIVO ASSEMBLY OF DNA VIA HOMOLOGOUS RECOMBINATION

(76) Inventors: Virginia Wood Cornish, New York, NY (US); Laura Michele Wingler, Chapel Hill, NC (US)

(21) Appl. No.: 13/282,195

(22) Filed: Oct. 26, 2011

Related U.S. Application Data
(63) Continuation of application No. PCT/US10/32962, filed on Apr. 29, 2010.
(60) Provisional application No. 61/174,272, filed on Apr. 30, 2009.

(51) Int. Cl. C12N 15/64; C12N 15/81; C12N 15/87

(52) U.S. Cl. 435/91.41; 435/440; 435/320.1

(57) ABSTRACT

According to the present invention, a DNA construct of interest is assembled from overlapping subfragments via an acceptor module which comprises the distal end of the construct at a position downstream from a promoter. The construct is assembled distal to proximal via homologous recombination events occurring in the span between that distal end of the construct and the upstream end of the promoter. These recombination events occur iteratively between the acceptor module and alternative donor modules. Successful recombination places one of at least two marker genes under the transcriptional control of an active form of the promoter. As a result of alternating use of two varieties of donor modules, as few as two selection markers may be used to produce a complex DNA construct.

### PRIORITY CLAIM

This application is a continuation application from PCT/US2010/032962, filed Apr. 29, 2010, which claims priority to U.S. Provisional Application No. 61/174,272, filed Apr. 30, 2009, the contents of which are incorporated by reference in their entireties herein.

### GRANT INFORMATION

This invention was made with government support under NIH Grant No. ROI GM62867 awarded by the National Institutes of Health. The government has certain rights in the invention.

### 1. INTRODUCTION

The present invention relates to methods and compositions which enable assembly of large and multiple DNA subfragments in vivo via homologous recombination.

### 2. BACKGROUND OF THE INVENTION

### 2.1 The Challenge of Cloning Large DNA Sequences

In order to functionally use information obtained from genome project initiatives for genetic engineering it is desirable to be able to construct and clone large tracts of sequence. For example, where the genes in an advantageous biosynthetic pathway are known, to be able to introduce that pathway into an organism nucleic acid encoding the component enzymes would need to be isolated, cloned, and propagated—most easily as a single construct that could be introduced into a progenitor cell of the organism. Examples where large-scale cloning has been successful exist, and typically required a work-intensive approach and substantial laboratory resources, including manpower. Cello et al., 2002, Science 297:1016 synthesized a full-length poliovirus complementary DNA (“cDNA”), thereby demonstrating that “it is possible to synthesize an infectious agent by in vitro biochemical means solely by following instructions from a written sequence.” Smith et al., 2003, Proc. Natl. Acad. Sci. U.S.A. 100:15440-15445 accomplished in vitro assembly of the complete infectious genome of bacteriophage φpX174 in fourteen days by building 5 kb “synthons” from short oligonucleotides and then assembling the synthons by conventional cloning methods. Kodumal et al., 2004, Proc. Natl. Acad. Sci. U.S.A. 101:15573-15578 describes a technique which, rather than assembling short oligonucleotides, generates 500-800 bp synthons by PCR which are joined into 5 kb fragments and then into longer DNA segments, and reports synthesis of a contiguous 32 kb polyketide synthase gene cluster. Reisinger et al., 2006, Nature Protocols 1:2596-2603 reports that a 32 kb DNA fragment was synthesized from 40-mer oligonucleotides via 500 bp synthons produced by a two-step polymerase chain reaction (“PCR”) followed by ligation-independent cloning. See also Pfeifer et al., 2001, Science 291:1790-1792; Ro et al., 2006, Nature 440:940-943; Martin et al., 2003, Nature Biotechnology 21:796-802; and DeJong et al., 2006, Biotechnol. Bioeng. 93:212-224.

More recently, cloning techniques have been developed that are practiced, at least in part, in vivo, which utilize natural processes, such as homologous recombination and selection, to increase efficiency. Itaya et al., 2005, Proc. Natl. Acad. Sci. U.S.A. 102:15971 cloned the 3.5 megabase (Mb) genome of Synechocystis PCC6803 into the 4.2 Mb genome of Bacillus subtilis using a technique known as “inchworm elongation” which utilizes a long (e.g. >100 kb) DNA template as a construct base. Subsequently, the same group reported using a method which does not require such a template but that rather uses homologous recombination between “domino clones” and an alternating marker system to assemble long stretches of DNA (e.g., originating in mouse mitochondrion and rice chloroplast) in the Bacillus subtilis genome.

### 2.2 Building DNA Constructs in Yeast

Strategies for genetic engineering have capitalized on the homologous recombination system in yeast and on the fact that double-strand DNA breaks are highly reactive substrates for homologous recombination (Ma et al., 1987, Gene 58:201-216 citing Orr-Weaver et al., 1981, Proc. Natl. Acad. Sci. U.S.A. 78:6354-6358; Orr-Weaver et al., 1983, Proc. Natl. Acad. Sci. U.S.A. 80:4417-4421). Such methodologies have been employed to manipulate the chromosomal loci of cloned genes (Ma et al., 1987, Gene 58:201-216 citing Winston et al., 1983, Methods Enzymol. 101:211-228) and to repair a linearized plasmid (Ma et al., 1987, Gene 58:201-216 citing Kunes et al., 1985, J. Mol. Biol. 184:375-387). Ma et al. (1987, Gene 58:201-216) extended the latter, co-transforming a linearized bacterial plasmid and a DNA fragment containing a selectable marker and plasmid-homologous regions to produce a new plasmid carrying the marker gene.

Subsequently, “linker-mediated assembly” was developed, which generates a bacterial plasmid containing a cloned insert in yeast by joining, via homologous recombination, introduced DNA fragments of interest via short, synthetic “recombination linkers” bearing regions of homology (Raymond et al., 1999, BioTechniques 26:134-141). The resulting plasmid is shuttled into Escherichia coli for subsequent screening and large-scale growth (Id.). This methodology was used to subclone inserts greater than 30 kb in size (Raymond et al., 2002, Genome Res. 12:190-197.

More recently, Hutchison's group has reported using homologous recombination in yeast to subclone overlapping fragments of DNA constituting an entire mycoplasmal genome. In a first report, four large, overlapping quarter-genome inserts (one containing plasmid sequence) were constructed in vitro, then transformed into yeast to generate the full 592 kb circular genome (Gibson et al., 2008, Science 319:1215-1220), A later paper reports one-step assembly of 25 overlapping fragments (each containing at least 80 bp of overlapping sequence) to re-create the mycoplasma genome in yeast (Gibson et al., 2008, Proc. Natl. Acad. Sci. 105:20404-20409). Success of the latter method depending upon a single yeast cell absorbing all 25 different fragments, translocating them to the nucleus, and successfully recombining them together (Id). After an initial selection step, clones were screened by multiplex polymerase chain reaction (PCR) to identify a clone containing the complete insert (Id.). Using an approach similar to that of Gibson et al., Zhao and co-workers reported successful assembly of up to a 19-kb, two-pathway biosynthetic cluster by co-transformation of 9 overlapping DNA fragments into yeast (Shao et al, 2009, Nucleic Acids Res. 37:e16).

### 3. SUMMARY OF THE INVENTION

The present invention relates to a method for preparing DNA constructs in vivo using homologous recombination, and compositions that may be used in such a method. It offers the advantage of requiring only a limited number of reagents and materials for the generation of complex DNA constructs. Important features include the creation of a double-stranded DNA break in the area targeted for recombination and the use of a limited set of selection markers, thereby promoting efficiency. The desired DNA construct is built incrementally by sequential homologous recombination events, and as such, the inventive method is alternatively referred to herein as “reiterative recombination.”

According to the present invention, a DNA construct of interest is assembled from overlapping subfragments via an acceptor module which comprises the distal end of the construct (where “distal” refers to a position downstream from a reference promoter). The construct is assembled distal to proximal (i.e., toward the promoter) via homologous recombination events occurring in the span between that distal end of the construct and the upstream end of the promoter. These recombination events occur iteratively between the acceptor module and alternative donor modules. Successful recombination places one of at least two marker genes under the transcriptional control of an active form of the promoter. As a result of alternating use of two varieties of donor modules, as few as two selection markers may be used to produce a complex DNA construct.

### 4. BRIEF DESCRIPTION OF THE FIGURES

FIG. 1. Schematic diagram of a preferred embodiment of the invention. A founder acceptor cell (1) containing a first subfragment of DNA construct to be assembled, an endonuclease 1 cleavage site (downward arrow) and a gene encoding a selection Marker 1 under the control of a conditional promoter (*) and its activating upstream activating sequence (UAS) is mated with a first donor cell (2) containing an inducible endonuclease 1 gene, a gene encoding a selectable marker S1 (under the control of an active promoter so that S1 is expressed in the first donor cell), and a second subfragment of the DNA construct to be assembled, an endonuclease 2 cleavage site (upward arrow), and a gene encoding a selection Marker 2 operably linked to a conditional promoter absent its activating UAS. The cells are then optionally selected for S1. After genetic exchange (mating or transformation) has occurred, endonuclease 1 expression is induced, resulting in a double strand break between the first DNA construct subfragment and the Marker 1 gene, which permits homologous recombination to occur between homologous regions in the first and second DNA subfragments and upstream of Marker genes 1 and 2. Selection for expression of Marker 2 identifies a first progeny acceptor cell (3), which now contains joined first and second DNA construct subfragments, an endonuclease 2 cleavage site (upward arrow), and a gene encoding Marker 2 operably linked to the conditional promoter (*) and its activating UAS. The first progeny acceptor cell is then mated with a second donor cell (4) containing an inducible endonuclease 2 gene, a gene encoding a selectable marker S2 (under the control of an active promoter so that S2 is expressed in the second donor cell (S1 and S2 may be the same but preferably are different)), a third subfragment of the DNA construct to be assembled, an endonuclease 1 cleavage site (downward arrow), and a gene encoding selection Marker 1 operably linked to a conditional promoter absent its activating UAS. The cells are then optionally selected for S2. After genetic exchange (mating or transformation) has occurred, endonuclease 2 expression is induced, resulting in a double strand break between the joined first and second DNA constructs and the gene encoding Marker 2, which permits homologous recombination to occur between homologous regions in the second and third DNA construct subfragments and upstream of Marker genes 2 and 1. Selection for expression of Marker 1 identifies a second progeny acceptor cell (5), which now contains joined first, second and third DNA construct subfragments, an endonuclease 1 cleavage site (downward arrow), and a gene encoding Marker 1 operably linked to the conditional promoter (*) and its activating UAS. This procedure may then optionally be repeated, first with a third donor cell containing an inducible endonuclease 1 gene, a fourth subfragment of the DNA construct, an endonuclease 2 cleavage site, and Marker 2, the progeny of which is mated with a fourth donor cell containing an inducible endonuclease 2 gene, a fifth subfragment of the DNA construct, an endonuclease 1 cleavage site and Marker 1, and alternating thereafter in an analogous pattern.

FIG. 2. Schematic diagram of a preferred embodiment of the invention, wherein a first endonuclease, operably linked to the galactose-inducible promoter pGAL, and a second endonuclease, operably linked to the tetracycline-inducible promoter tet07, as well as the TetR′-VP16 gene are comprised in acceptor host cell DNA. A founder acceptor cell (1) containing the founder acceptor module (2) is mated (undergoes genetic exchange) (16) with an odd donor cell (3) containing (or the founder acceptor cell is transformed with DNA comprising) an odd donor complex (4) comprising the URA3 selection marker and an odd donor module (5). In the progeny cell (6), endonuclease 1 expression is induced with galactose, and the endonuclease makes a double strand break in the founder acceptor module, which facilitates homologous recombination between the DNA subfragments to be assembled (8) and upstream of the selection marker HIS3 (9) which is replaced by the selection marker LEU2. Selection for LEU2 expression (10) as well as resistance to 5-fluoroorotic acid (FOA^{R}) selects progeny acceptor cell (11) containing the progeny acceptor module (12). The progeny acceptor cell then undergoes genetic exchange (17) with an even donor cell containing (13) (or the progeny acceptor cell is transformed with DNA comprising) an even donor complex (14) comprising an even donor module (15). In the progeny cell (18), endonuclease 2 expression is induced with doxycycline, and the endonuclease makes a double strand break in the progeny acceptor module, which facilitates homologous recombination between the DNA subfragments to be assembled (20) and upstream of the selection marker LEU2 (21), which is replaced by selection marker HIS3. Selecting for HIS3 expression (22) selects progeny acceptor cell (23) containing the progeny acceptor module (24).

FIG. 3. Diagram showing founder acceptor cell (1) containing chromosomally integrated Endonuclease genes 1 and 2, each under the control of an inducible promoter (25), as well as a chromosomally integrated founder acceptor module (2) and first odd donor complex (4).

FIG. 4. Diagram showing construction of donor cassettes.

FIG. 5. Flow chart showing scheme for strain construction.

FIG. 6. Diagram showing construct to be assembled from subfragments A-D, where subfragments can be prepared based on primers (solid lines with arrowheads) of synthetic oligonucleotides to produce subfragments with overlapping ends. The terminal primers at the boundaries of the construct may contain sequences (* and that provide for insertion at a restriction site for cloning.

FIG. 7. Diagram showing a non-limiting embodiment of the system of the invention.

FIG. 8A-D. (A) Conversion of an acceptor module into a donor module. (B) (B) Schematic for building a DNA construct using convergent reiterative recombination. (C) Scheme for using convergent reiterative recombination to reconstruct the epothilone gene cluster. (D) Depiction of a generic convergent acceptor module. “A” and “D” represent constructs used as acceptors and donors, respectively. Lines in ORFs indicate PKS and NRPS modules.

FIG. 9. Construction of the parental reiterative recombination strain for sequential incorporation of lacZ, gusA and MET15 into yeast.

FIG. 10A-E. Reiterative recombination system. (A) Details of the assembly process for the system, in which lacZ, gusA, and MET15 were sequentially integrated into the chromosome. (B) All donor plasmids were constructed by plasmid gap repair, in which a digested donor plasmid (with no features specific to the construct being built) and PCR fragments containing appropriate homology regions were co-transformed into the reiterative recombination host strain and assembled via homologous recombination. (C) The results of the endonuclease induction step from round 2 are shown as a representative example. As negative controls, cells containing identical donor plasmids lacking the SceI endonuclease gene and/or the gusA fragment with lacZ homology were induced in parallel. A calculated 6×10⁶ cells were plated on SC(-Histidine) media to assay for selective marker conversion after a 12-hour galactose induction. Parallel inductions in glucose media generated marker switching rates comparable to the galactose negative controls. (D) Phenotypes of 12 unique colonies from each round of assembly that are cured of donor plasmids. In columns, recombinants are assayed for the HIS3 (SC(-Histidine)) and LEU2 (SC(-Leucine)) markers. In rows, Magenta-Gal (5-Bromo-6-chloro-3-indolyl-β-D-galactopyranoside), X-Gluc (5-bromo-4-chloro-3-indolyl-beta-D-glucuronic acid), and SC(-Methionine) media were used to assay for lacZ, gusA, and MET15, respectively. (E) PCR and restriction analysis of genomic DNA from 4 unique colonies from round 3 of the marker proof-of-principle system. All colonies gave the expected amplicons and restriction fragments. (P=undigested PCR product, B=BfuAI digest, W=BsaWI digest, G=BsrGI digest, G/H=BsrGI/HindIII digest, A=BsmAI digest).

FIG. 11A-D. (A) The carotenoid biosynthetic cluster (5 kb) assembled using reiterative recombination; each colored fragment was added in a different round of elongation for a total of 4 rounds. (B) E. coli cells transformed with a plasmid containing the carotenoid biosynthetic pathway assembled by reiterative recombination (bottom) have the same colorimetric phenotype as cells containing the original plasmid constructed by the Arnold laboratory (top left). (C and D) Features of donor plasmids and an E. coli/S. cerevisiae (shuttle) acceptor plasmid.

FIG. 12A-B. (A) Genes for lycopene production assembled into the chromosome via reiterative recombination to date (not to scale; order has been revised since initial grant submission). Solid lines and different colored boxes indicate junctions created by endonuclease-stimulated recombination; dashed lines indicate junctions created during plasmid gap repair following transformation. The TRP1 and MET15 markers separate all repeated elements. Numbers and arrows indicate amplicons in the PCR analysis below. (B) Representative data from the PCR and restriction analysis of genomic DNA from a randomly selected colony from round 7. All colonies gave the expected amplicons and restriction fragments.

### 5. DETAILED DESCRIPTION OF THE INVENTION

The present invention is based upon a system comprising the following four basic elements:

(i) an acceptor module comprising, downstream to upstream (where transcription proceeds upstream to downstream, i.e., a coding sequence is downstream of the promoter element that controls the transcription of the coding sequence), (a) a first DNA subfragment that is to be assembled to form a construct of interest; (b) an endonuclease cleavage site; and (c) a gene encoding a first selectable marker operably linked to (d) an active promoter or active promoter complex;

(ii) a first donor module (referred to as the “odd donor module” herein) comprising, downstream to upstream, (a) a second DNA subfragment, that is to be joined to the first DNA subfragment of (i) and that shares a region of homology with it; (b) an endonuclease cleavage site; (c) a gene encoding a second selectable marker (which is not the same as the first selectable marker); and (d) a region upstream of the gene of (c) which is homologous to a region of the acceptor module such that recombination between the acceptor module and the first donor module in this region places the gene encoding the second selectable marker under transcriptional control of the active promoter or active promoter complex and thereby inactivates the gene encoding the first selectable marker;

(iii) a second donor module (referred to as the “even donor module” herein) comprising, downstream to upstream, (a) a third DNA subfragment, that is to be joined to the second DNA subfragment of (ii) and that shares a region of homology with it; (b) an endonuclease cleavage site; (c) a gene encoding the first selectable marker; and (d) a region upstream of the gene of (c) which is homologous to a region of the acceptor module such that recombination between the acceptor module and the first donor module in this region places the gene encoding the first selectable marker under transcriptional control of the active promoter or promoter complex and inactivates the gene encoding the second selectable marker;

(iv) a host cell in which homologous recombination occurs or can be made to occur, which serves as a host for the acceptor module in which the DNA construct is built (“the acceptor cell”);

(v) a plurality of host cells, capable of genetic exchange with the acceptor cell, which serve as hosts for donor modules (donor cells); and

(vi) one or more nucleic acid encoding an endonuclease, a cleavage site of which is the site (b) present in the acceptor module and at least one donor module, operably linked to a promoter.

Successive overlapping subfragments of the DNA construct are inserted into alternating odd and even donor modules. The DNA construct is assembled by promoting endonuclease cleavage of the acceptor module, providing conditions that allow homologous recombination between the acceptor module and a donor module, and then selecting for acceptor cells containing a progeny acceptor module in which the marker has switched, indicative that homologous recombination has successfully occurred.

For clarity, and not by way of limitation, the detailed description is divided into the following subsections:

(i) acceptor modules;

(ii) donor modules;

(iii) host cells;

(iv) shuttle vectors;

(v) methods;

(vi) kits;

(vii) convergent reinterative recombination; and

(viii-x) prophetic examples.

### 5.1 Acceptor Modules

The present invention provides for an acceptor module comprising, downstream to upstream, (i) a DNA subfragment that is to be assembled to form a construct of interest; (ii) an endonuclease cleavage site; and (iii) a gene encoding a selectable marker operably linked to (iv) an active promoter or promoter complex. The species of selectable marker and optionally the endonuclease cleavage site switches back and forth between alternative embodiments as successive DNA subfragments are added.

A “founder acceptor module” is an acceptor module in a founder acceptor cell which is not the product of recombination between a precursor acceptor module and a donor module.

A “progeny acceptor module” is an acceptor module in a progeny acceptor cell which is the product of recombination between a precursor acceptor module and a donor module.

In certain non-limiting embodiments, the acceptor module preferably is comprised integrated into chromosomal DNA. In other non-limiting embodiments, the acceptor module is comprised in non-chromosomal DNA, such as a plasmid.

The DNA construct to be assembled may be any DNA of interest. As non-limiting examples, the DNA construct may comprise genes encoding enzymatic components of a biosynthetic pathway or a metabolic pathway, an organism genome, or a genetic circuit for use in a synthetic biological system. As repeated elements may interfere with a scheme involving homologous recombination, it is desirable to design away from the presence of such elements where possible.

The endonuclease cleavage site is a site having a nucleic acid sequence which is cleaved by a nuclease present in (and optionally introduced into) a founder or progeny acceptor cell. As a first, non-limiting example, the endonuclease is the HO endonuclease, which makes a double strand break (shown by arrows) resulting in a 4 bp single-stranded overhang, in the complementary sequences 5′ CCGCAACA↓GTAA 3′(SEQ ID NO:1) and 3′GGCG↑TTGTCATT 5′ (SEQ ID NO:2) (Nickoloff et al., 1990, Mol. Cell. Biol. 10:1174-1179; Nickoloff et al., 1986, Proc. Natl. Acad. Sci. U.S.A. 83:7831-7835); accordingly, the endonuclease cleavage site in the acceptor module may comprise at least the sequence TXXXCGCAXCAXT (SEQ ID NO:3, where X are variable nucleotides), for example, but not by way of limitation, for the HO endonuclease cleavage site in the MATalpha allele, the cleavage site may comprise the sequence GGACTACTTCGCGCAACAGTATAA (SEQ ID NO: 4) or a sequence which is at least 80% or at least 90% homologous thereto and, for the HO endonuclease cleavage site in the MATa allele, the cleavage site may comprise the sequence TTTCAGCTTTCCGCAACAGTAAAA (SEQUENCE ID NO:5) or a sequence which is at least 80% or at least 90% homologous thereto. As a second, non-limiting example, the endonuclease is SceI, which makes a double strand break (shown by arrows) resulting in a 4 bp overhang, in the complementary sequences 5′ TAGGGATAA↓CAGGGTAAT 3′ (SEQ ID NO:6) and 3′ ATCCC↑TATTGTCCCATTA 5′ (SEQ ID NO:7) (Colleaux et al., 1988, Proc. Narl. Acad. Sci. U.S.A. 85:6022-6026) accordingly, the endonuclease cleavage site in the acceptor module may comprise at least the sequence TAGGGATAACAGGGTAAT (SEQ ID NO: 6) or a sequence which is at least 80% or at least 90% homologous thereto. As a third, non-limiting example, the endonuclease is the DmoI variant, which makes a double strand break (shown by arrows) resulting in a 4 bp single stranded overhang, in the complementary sequences 5′ GCCTTGCCGGGTAA↓GTTCCGGCGCG 3′(SEQ ID NO:8) and 3′ CGGAACGGCC↑CATTCAAGGCCGCGC 5′ (SEQ ID NO:9) (Dalgaard et al., 1994, J. Biol. Chem. 269: 28885); accordingly, the endonuclease cleavage site in the acceptor module may comprise at least the sequence 5′GCCTTGCCGGGTAA↓GTTCCGGCGCG 3′(SEQ ID NO:8) or a sequence which is at least 80% or at least 90% homologous thereto. Other endonucleases which may be used, with their respective cleavage sites, include, but are not limited to, PpoI (Lowery et al., 1992, Promega Notes 38; Argast et al., 1998, J. Mol. Biol. 280(3): 345-353); I-CreI (Arnould et al., 2006, J. Mol. Biol. 355(3):443-458); I-AniI (Y. Ho, et al., 1997, Proc. Natl. Acad. Sci. USA 94: 8994-8999; recognition sequence Scalley-Kim et al., 2007, J. Mol. Biol. 372:1305), and see Belfort and Roberts, 1997, Nucl. Acids Res. 25 (17): 3379, and Stoddard, 2005, Quarterly Review of Biophysics 38(1): 49.

In preferred, non-limiting embodiments of the invention, in the acceptor module, the endonuclease site is positioned so that the cleavage occurs immediately adjacent to, or within up to about 100 bp, or within up to about 500 bp, or within up to about 5 kb, or within up to about 10 kb of each site where homologous recombination is desired to occur (i.e., within the DNA construct subfragments and upstream of the selection marker).

The selectable marker may be any selectable marker known in the art and may be chosen based on a variety of standard criteria, including the organism which serves as acceptor/donor cell. For example, where the acceptor/donor cells are yeast, a selectable marker may be chosen from, for example but not by way of limitation, URA3, HIS3, LEU2, TRP1 and LYS2, which complement specific auxotrophic mutations in yeast, such as ura3 (e.g. ura3-52), his3 (e.g. his3delta1), leu2 (e.g. leu2delta1), trp1 (e.g. trp1delta1) and lys2 (e.g. lys2delta-202), as well as ADE1/ade1, ADE2/ade2, MET15/met15, KanMX for geneticin (G418) resistance, hygromycin B resistance, phleomycin resistance, etc. Preferred selection markers are those that permit both selection and counter-selection, for example, but not limited to, URA3 or LYS2. Where the acceptor/donor organism is a bacterium, the selection marker may be, for example but not by way of limitation, an antibiotic resistance gene, for example, but not by way of limitation, conferring resistance to ampicillin, neomycin, tetracycline, chloramphenicol, phleomycin, kanamycin, and spectinomycin.

The acceptor module further comprises an active promoter or active promoter complex. Active promoters are known in the art and may be selected based on the host organism. The active promoter may be an inducible promoter provided that the inducing agent is present when the promoter is desired to be active. A promoter complex is a plurality (i.e., at least two) operably linked sequences which together have significant promoter activity, for example, a promoter plus an upstream activating sequence (“UAS”), where said promoter is conditionally active when it is within a certain proximity of the UAS. Non-limiting examples of yeast promoters include plasma membrane H-ATPase (“PMA1”), the galactokinase gene promoter (“GAL1”), the alcohol dehydrogenase 2 promoter (“ADH2”), the translational elongation factor EF-1 alpha promoter (“TEF1”), the cytochrome c, isoform 1 promoter (“CYC1), the glycerol-3-phosphate dehydrogenase promoter (“GPD”), and the MET25 promoter. A non-limiting example of a promoter complex is the yeast pyruvate kinase gene (“PYK”) promoter, which is active when operably linked to an upstream activating sequence (“UAS”) and can optionally be repressed by an upstream repressible sequence. For example, but not by way of limitation, the PYK promoter may have the sequence of GenBank Ace. No. U12980.3 from nucleotide 68564 to 69195, and its correlate UAS may have the sequence of GenBank Acc. No. U12980.3 from nucleotide 68388 to 68563 or to 68544 or to 68563 (Nishizawa et al., 1989, Mol. Cell. Biol. 9(2):442) Another non-limiting example of a promoter complex is the yeast phosphogylcerate kinase gene (“PGK”) promoter, which is active when operably linked to an upstream activating sequence (“UAS”) and can optionally be repressed by an upstream repressible sequence. For example, but not by way of limitation, the PGK promoter may have the sequence of GenBank Acc. No. X59720.2 from nucleotide 137328 to 137739 and its correlate UAS may have the sequence of GenBank Acc. No. X59720.2 from nucleotide 137256 to 137327. Non-limiting examples of bacterial promoters include the T7 promoter, the LAC4 promoter, the trp-lac (“Tac”) promoter and the Arabinose promoter (pBAD).

### 5.2 Donor Modules

The present invention provides for a basic donor module which comprises, downstream to upstream, (i) a DNA subfragment of the construct to be assembled; (ii) an endonuclease cleavage site; (iii) a gene encoding a selectable marker; and (iv) a region upstream of the gene of (iii) which is homologous to a region of the acceptor module, such that recombination between the acceptor module and the first donor module in this region places the gene encoding the second selectable marker under transcriptional control of the active promoter or promoter complex. Of note, the structure of the acceptor module changes at successive stages of the assembly process, so that the homologous region of the acceptor molecule referred to below is either in the founder acceptor module (present in the founder acceptor cell) or is in a progeny acceptor module (in a progeny acceptor cell) that results from recombination with a donor module.

In particular non-limiting embodiments, the present invention provides for a pair of donor modules, the use of which is alternated. Said pair comprises an “odd donor module” and an “even donor module”, wherein (relative to a reference acceptor module):

the “odd donor module” comprises, downstream to upstream, (i) a second DNA subfragment, that is to be joined to a first DNA subfragment in an acceptor module and that shares a region of homology with said first DNA subfragment; (ii) an endonuclease cleavage site; (iii) a gene encoding a second selectable marker which differs from a gene for a first selectable marker in the acceptor module; and (iv) a region upstream of the gene of (iii) which is homologous to a region of the acceptor module such that recombination between the acceptor module and the odd donor module in this region places the gene encoding the second selectable marker under transcriptional control of the active promoter or promoter complex and inactivates the gene encoding the first selectable marker; and

the “even donor module” comprises, downstream to upstream, (i) a third DNA subfragment, that is to be joined to the second DNA subfragment of the odd donor module and that shares a region of homology with it; (ii) an endonuclease cleavage site; (iii) a gene encoding the first selectable marker; and (iv) a region upstream of the gene of (iii) which is homologous to a region of the acceptor module such that recombination between the acceptor module and the first donor module in this region places the gene encoding the first selectable marker under transcriptional control of the active promoter or promoter complex and inactivates the gene encoding the second selectable marker.

In certain preferable non-limiting embodiments, the donor module is comprised in non-chromosomal DNA, for example, a plasmid. In other non-limiting embodiments, the donor module is comprised in chromosomal DNA.

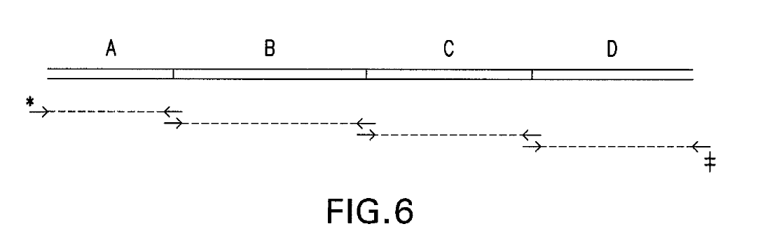

In a donor module, the DNA subfragment of the construct to be assembled comprises a region homologous to (for example, overlaps) the subfragment present in the acceptor module which is at least about 30, or at least about 40, or at least about 50, or at least about 60, or at least about 70, or at least about 85, or at least about 100 bp in length, positioned such that homologous recombination between the donor and acceptor subfragments extends the acceptor subfragment in the upstream direction. In said donor module, the DNA subfragment also comprises a region homologous to (for example, overlaps) a subfragment present in the next successive donor module to be used (e.g., the subfragment in an odd donor module overlaps with the subfragment in the next even donor module to be used). Again said region of overlap/homology is at least about 30, or at least about 40, or at least about 50, or at least about 60, or at least about 70, or at least about 85, or at least about 100 bp in length, or at least about 500 bp in length, or at least about 2000 bp in length. Homology, as that term is used herein, indicates sufficient sequence homology to permit homologous recombination to occur, for example, at least about 80 percent, at least about 90 percent, at least about 95 percent, or at least about 98 percent homology, as determined by standard software such as BLAST or FASTA, where a region having sequence identity is preferred. Such regions of homology may be produced by designing primers for synthesis of the subfragments (e.g., by PCR) which create regions of homology/overlap. An example of such design is shown in FIG. 6.

The endonuclease cleavage site is a site having a nucleic acid sequence which is cleaved by a nuclease present in (and optionally introduced into) a founder or progeny acceptor cell. As a first, non-limiting example, the endonuclease is the HO endonuclease, which makes a double strand break (shown by arrows) resulting in a 4 bp single-stranded overhang, in the complementary sequences 5′ CCGCAACA↓GTAA 3′(SEQ ID NO:1) and 3′GGCG↑TTGTCATT 5′ (SEQ ID NO:2) (Nickoloff et al., 1990, Mol. Cell Biol. 10:1174-1179; Nickoloff et al., 1986, Proc. Natl. Acad. Sci. U.S.A. 83:7831-7835); accordingly, the endonuclease cleavage site in the acceptor module may comprise at least the sequence TXXXCGCAXCAXT (SEQ ID NO:3, where X are variable nucleotides), for example, but not by way of limitation, for the HO endonuclease cleavage site in the MATalpha allele, the cleavage site may comprise the sequence GGACTACTTCGCGCAACAGTATAA (SEQ ID NO: 4) or a sequence which is at least 80% or at least 90% homologous thereto, and, for the HO endonuclease cleavage site in the MATa allele, the cleavage site may comprise the sequence TTTCAGCTTTCCGCAACAGTAAAA (SEQUENCE ID NO:5) or a sequence which is at least 80% or at least 90% homologous thereto. As a second, non-limiting example, the endonuclease is SceI, which makes a double strand break (shown by arrows) resulting in a 4 bp overhang, in the complementary sequences 5′ TAGGGATAA↓CAGGGTAAT 3′ (SEQ ID NO:6) and 3′ ATCCC↑TATTGTCCCATTA 5′ (SEQ ID NO:7) (Colleaux et al., 1988, Proc. Natl. Acad. Sci. U.S.A. 85:6022-6026) accordingly, the endonuclease cleavage site in the acceptor module may comprise at least the sequence TAGGGATAACAGGGTAAT (SEQ ID NO: 6) or a sequence which is at least 80% or at least 90% homologous thereto. As a third, non-limiting example, the endonuclease is the DmoI variant, which makes a double strand break (shown by arrows) resulting in a 4 bp single stranded overhang, in the complementary sequences 5′ GCCTTGCCGGGTAA↓GTTCCGGCGCG 3′(SEQ ID NO:8) and 3′ CGGAACGGCC↑CATTCAAGGCCGCGC 5′ (SEQ ID NO:9) (Dalgaard et al., 1994, J. Biol. Chem. 269: 28885); accordingly, the endonuclease cleavage site in the acceptor module may comprise at least the sequence 5′ GCCTTGCCGGGTAA↓GTTCCGGCGCG 3′(SEQ ID NO:8) or a sequence which is at least 80% or at least 90% homologous thereto. Other endonucleases which may be used, with their respective cleavage sites, include, but are not limited to, PpoI (Lowery et al., 1992, Promega Notes 38; Argast et al., 1998, J. Mol. Biol. 280(3): 345-353); I-CreI (Arnould et al., 2006, J Mol. Biol. 355(3):443-458); I-Anil (Y. Ho, et al., 1997, Proc. Natl Acad. Sci. USA 94: 8994-8999; recognition sequence Scalley-Kim et al., 2007, J. Mol. Biol. 372:1305), and see Belfort and Roberts, 1997, Nucl. Acids Res. 25 (17): 3379 and Stoddard, 2005, Quarterly Review of Biophysics 38(1): 49.

In certain preferred non-limiting embodiments, the endonuclease cleavage site in the donor molecule is cleaved by an endonuclease different from the endonuclease that cleaves the correlate acceptor module. So, as one non-limiting example, where an acceptor cell carries an acceptor module that comprises a cleavage site for HO endonuclease, the acceptor cell may be mated to a donor cell that carries a donor module that comprises a cleavage site for SceI endonuclease.

In certain preferred non-limiting embodiments, the endonuclease cleavage site in the odd donor module is cleaved by an endonuclease different from the endonuclease that cleaves the even donor module. As one non-limiting embodiment, odd donor modules may comprise a cleavage site for HO endonuclease, and even donor modules may comprise a cleavage site for SceI endonuclease, or vice-versa.

In preferred, non-limiting embodiments of the invention, in the donor module, the endonuclease site is positioned so that the cleavage occurs immediately adjacent to, or within up to about 100 bp, or within up to about 500 bp, or within up to about 5 kb, or within up to about 10 kb of each site where homologous recombination is desired to occur (i.e., within the DNA construct subfragments and upstream of the selection marker).

The selectable marker may be any selectable marker known in the art and may be selected based on a variety of standard criteria, including the organism which serves as acceptor/donor cell. For example, where the acceptor/donor cells are yeast, a selectable marker may be chosen from, for example but not by way of limitation, URA3, HIS3, LEU2, TRP1 and LYS2, which complement specific auxotrophic mutations in yeast, such as ura3 (e.g. ura3-52), his3 (e.g. his3delta1), leu2 (e.g. leu2delta1), trp1 (e.g. trp1delta1) and lys2 (e.g. lys2delta-202), as well as ADE1/ade1, ADE2/ade2, MET15/met15, KanMX for geneticin (G418) resistance, hygromycin B resistance, phleomycin resistance, etc. Preferred selection markers are those that permit both selection and counter-selection, for example, but not limited to, URA3 or LYS2. Where the acceptor/donor organism is a bacterium, the selection marker may be, for example but not by way of limitation, an antibiotic resistance gene, for example, but not by way of limitation, conferring resistance to ampicillin, neomycin, tetracycline, chloramphenicol, phleomycin, kanamycin and spectinomycin. For a donor module to be genetically recombined with a given acceptor module, the selection markers comprised in the donor and acceptor modules are not the same. In non-limiting embodiments of the invention, the selection marker of odd donor modules is different from the selection marker of even donor modules.

Upstream of the gene encoding the selectable marker in the donor module, there is a region of homology shared with a region upstream of the selectable marker in the acceptor module with which said donor module is to be recombined, said region of homology being at least about 30, or at least about 40, or at least about 50, or at least about 60, or at least about 70, or at least about 85, or at least about 100 bp in length, or at least about 500 bp, or at least about 2000 bp. Similarly, there is a region of homology, which may or may not overlap with that region referred to in the preceding sentence, between a region upstream of the selectable marker in a given donor module and the next donor module which is to be recombined with it—for example, between an odd donor module and the next even donor module to be used, said region of homology being at least about 30, or at least about 40, or at least about 50, or at least about 60, or at least about 70, or at least about 85, or at least about 100 bp in length. In specific, non-limiting embodiments of the invention, either or both of said donor module regions may occur upstream of the selectable marker but downstream of the promoter region or upstream of the selectable marker and within the promoter or promoter complex region. For example, but not by way of limitation, there is no active promoter or promoter complex in the donor module, such that the selection marker in the donor module is not expressed or is expressed at a much lower level than the selection marker in the acceptor cassette.

In certain preferred non-limiting embodiments, the donor module is comprised within a donor complex (for example, a plasmid) which further comprises a gene encoding an endonuclease under the control of an inducible promoter (a promoter that may be directly induced by an inducing agent or indirectly induced by inhibition of a repressor element). A donor complex is termed an “odd” or “even” complex according to whether the contained module is odd or even where an “odd donor complex” comprises an “odd donor module”, etc. Said endonuclease gene and its promoter are positioned outside of the donor module, that is to say, outside the region including and between the DNA subfragment to be assembled, the endonuclease cleavage site, the gene encoding the marker and its upstream region homologous to the acceptor module (in other words, it is outside the region that enters the acceptor module via homologous recombination). Non-limiting examples of endonucleases which may be used (Prieto, et al., 2008, include HO endonuclease (Russell et al., 1986, Mol. Cell. Biol. 6(12):4281), SceI endonuclease (Plessis et al., 1992, Genetics 130(3): 451), and the DmoI variant endonuclease Prieto, et al. J. Biol. Chem. 283 (7): 4364). In a specific non-limiting embodiment, an odd donor complex comprises a gene for a first endonuclease, under the control of an inducible promoter, where said first endonuclease cleaves at the endonuclease cleavage site of the corresponding acceptor module and the next even donor module, but not at the endonuclease cleavage site comprised in the odd donor module carried by the odd donor complex itself. In a related specific non-limiting embodiment, an even donor complex comprises a gene for a second endonuclease, under the control of an inducible promoter, where said second endonuclease cleaves at the endonuclease cleavage site of the corresponding odd donor module but not at the endonuclease cleavage site comprised in the even donor module or carried by the even donor complex itself. The inducible promoters of the odd and even donor molecules may be the same and/or be induced by the same agent, of they may be different and induced by different agents. Non-limiting examples of inducible promoters include the GAL1 promoter (Johnston and Davis, 1984, Mol. Cell. Biol. 4: 1440) and the tetracycline-inducible promoter (e.g., the “tet07 promoter” or the “tet02” promoter; Belli, et al., 2004, Nucl. Acids Res. 26: 942), the CUP1 promoter, (Etcheverry, 1990, T. Meth. Enzymol. 185: 31); the MET25 promoter (Sangsoda et al., 1985, Mol. Gen. Genet. 200: 407; the (forward) tetracycline system (Gari et al., 1997, Yeast, 13: 837) and the repressible CTR1 and CTR3 promoters (Labbe et al., 1997, J. Biol. Chem. 272: 15951).

In further non-limiting embodiments, the donor complex further comprises a third selectable marker different from those markers comprised in the acceptor and donor modules that are shuttled into and out of the acceptor module, said third selectable marker operably linked to a promoter molecule which may be constitutively active or inducible. Said gene (and its promoter) is positioned outside of the donor module—the region comprising and between the DNA subfragment to be assembled, the endonuclease cleavage site, the gene encoding the marker and its upstream region homologous to the acceptor module (in other words, it is outside the region that enters the acceptor module via homologous recombination). Said selectable marker may be used to select for donor host cells that have successfully incorporated the donor module, as well as progeny host cells that comprise donor host cell genetic material. For example, where the acceptor/donor cells are yeast, a selectable marker may be chosen from, for example but not by way of limitation, URA3, HIS3, LEU2, TRP1 and LYS2, which complement specific auxotrophic mutations in yeast, such as ura3 (e.g. ura3-52), his3 (e.g. his3delta1), leu2 (e.g. leu2delta1), trp1 (e.g. trp1delta1) and lys2 (e.g. lys2delta-202), as well as ADE1/ade1, ADE2/ade2, MET15/met15, KanMX for geneticin (G418) resistance, hygromycin B resistance, phleomycin resistance, etc. Preferred selection markers are those that permit both selection and counter-selection, for example, but not limited to, URA3 or LYS2. Where the acceptor/donor organism is a bacterium, the selection marker may be, for example but not by way of limitation, an antibiotic resistance gene, for example, but not by way of limitation, conferring resistance to ampicillin, neomycin, tetracycline, chloramphenicol, phleomycin, kanamycin and spectinomycin.

### 5.3 Host Cells

The present invention provides for (i) a host cell in which homologous recombination occurs or can be made to occur, which serves as a host for the acceptor module in which the DNA construct is built (“the acceptor cell”); and (ii) a plurality of host cells, capable of genetic exchange with the acceptor host cell, which serve as hosts for donor modules (donor cells).

“Genetic exchange” refers to a comingling of genetic material such as occurs with mating or conjugation or transformation (or similar techniques).

The present invention provides for a founder acceptor cell which contains a founder acceptor module.

The present invention provides for a progeny acceptor cell which contains a progeny acceptor module. It should be noted that when an acceptor and donor cell mate, but before homologous recombination has occurred, the cell is simply referred to as a “progeny cell” but it is not a progeny acceptor cell.

The present invention provides for an odd donor cell and an even donor cell (depending upon whether the contained module is odd or even; an odd donor cell carries an odd donor module).

The host cell comprises or is caused to comprise (for example, by introduction of a donor module) one or more nucleic acids encoding one or more endonucleases, each preferably operably linked to an inducible promoter. Where different endonuclease genes are present, they are preferably, but not by limitation, operably linked to promoters inducible by different agents. Said endonuclease gene(s) may be integrated into a chromosome or may be extra-chromosomal (e.g., in a plasmid). Suitable endonucleases include, but are not limited to, HO endonuclease, SceI endonuclease, DmoI variant endonuclease. Non-limiting examples of inducible promoters include the gall promoter and the tetracycline-inducible promoter (e.g., the “tet07 promoter”) and others listed above.

In a preferred embodiment, the host cell(s) are yeast cells. Suitable yeast include, but are not limited to, Saccharomyces cerevisiae, Pichia pastoris, and Schizosaccharomyces pombe. In other non-limiting embodiments, the host cell may be a bacterial cell, a slime mold cell, a fungal cell, an algae cell, an animal cell, etc. For example, but not by way of limitation, the host cell may be Bacillus subtilis.

In a specific, non-limiting example, the host cell may be a yeast cell, e.g. a Saccharomyces cerevisiae cell, with non-cleavable MAT alleles to avoid homozygous diploids. For example, and without being bound by any particular theory, in a system where the inducible HO endonuclease is employed, cleavage of MAT in a diploid (a/α) can lead to mating type switching at one allele, resulting in a homozygous diploid genotype (a/a or α/α). These homozygous diploids can potentially mate with another haploid (α or a, respectively) or another homozygous diploid, leading to polyploid cells (Herskowitz and R. Jensen, 1991, Method. Enzymol. 194: 132).

### 5.4 Shuttle Vectors

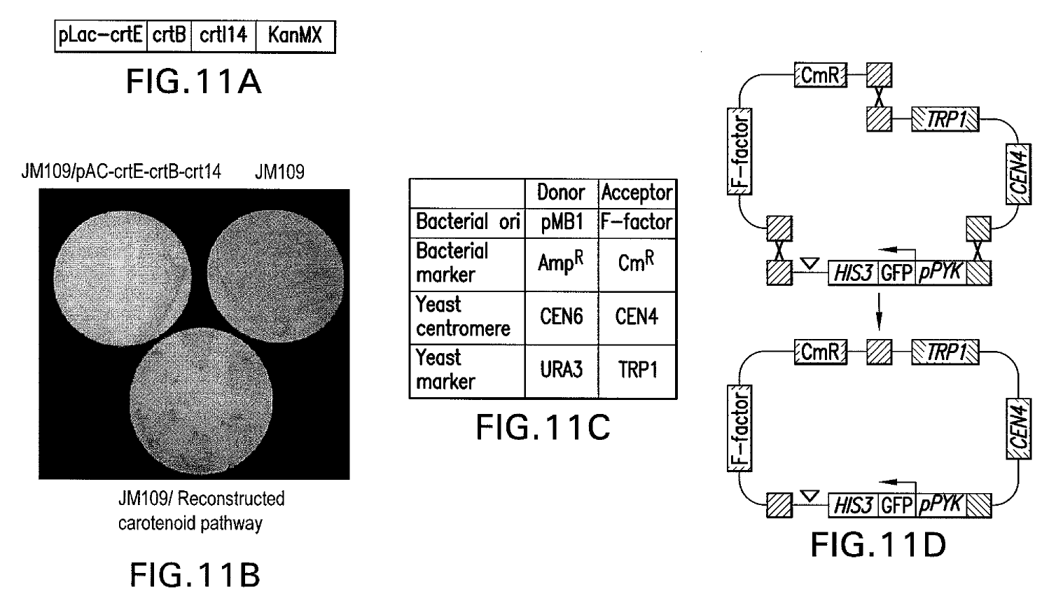

Once the DNA construct is assembled, it may be desirable to express as protein one or more genes contained within the DNA construct. Expression may be performed by the host organism in which reiterative recombination was performed (e.g., Saccharomyces cereviseae), or, alternatively, it may be desirable to use another organism to express the gene or genes to produce a protein or proteins of interest; suitable organisms are known in the art and include, but are not limited to, Escherichia coli, Streptomyces coelicolor, and Streptomyces lividans. Accordingly, the present invention provides for modules that may be used to permit shuttling of the DNA construct resulting from reiterative recombination in a first organism into a second organism This may be facilitated by including, in the acceptor module, one or more elements that allow for replication and/or selection in the (second) organism in which expression is to occur and/or cleavage sites for excision of the gene or genes to be expressed. For example, where reiterative recombination is performed in yeast but expression in a bacterium is desired, a shuttle acceptor molecule may comprise one or more of an origin of replication utilized in the bacterium, a selection marker for the bacterium (e.g., an antibiotic resistance gene), and/or restriction endonuclease cleavage sites on either side of the gene or genes to be expressed. As one specific, non-limiting example, a shuttle construct may be prepared by introducing a F replicon-CmR restriction fragment (e.g. from pBeloBAC11 (New England Biolabs)) into the acceptor module. See, for example but not by way of limitation, FIGS. 11C and 11D.

Accordingly, in particular non-limiting embodiments, the present invention provides for a shuttle acceptor module that may be used to assemble a DNA construct in a yeast, where a gene or genes of the assembled DNA construct are to be expressed in a bacterium, comprising downstream to upstream (a) a first DNA subfragment that is to be assembled to form the construct; (b) an endonuclease cleavage site; and (c) a gene encoding a first selectable marker operably linked to (d) an active promoter or active promoter complex, and (e) one or more elements selected from the group consisting of an origin of replication utilized in the bacterium, a selection marker for the bacterium, and/or restriction endonuclease cleavage sites on either side of the gene or genes to be expressed.

### 5.5 Methods

According to the invention, successive overlapping subfragments of the DNA construct are inserted into alternating odd and even donor modules. The DNA construct is assembled by allowing genetic exchange between acceptor and donor host cells, promoting endonuclease cleavage of the acceptor module, providing conditions that allow homologous recombination, and then selecting for acceptor host cells in which the marker has switched, indicative that homologous recombination has successfully occurred. In particular, the DNA construct is assembled, downstream to upstream, by successively exchanging overlapping construct fragments, with each successive fragment extending the construct in the upstream direction, via homologous recombination triggered by the action of a site-specific endonuclease on a site between the growing construct and an active promoter or promoter complex, where successful homologous recombination is detected by the switch of a selection marker operably linked to said promoter/promoter complex from one to another alternative.

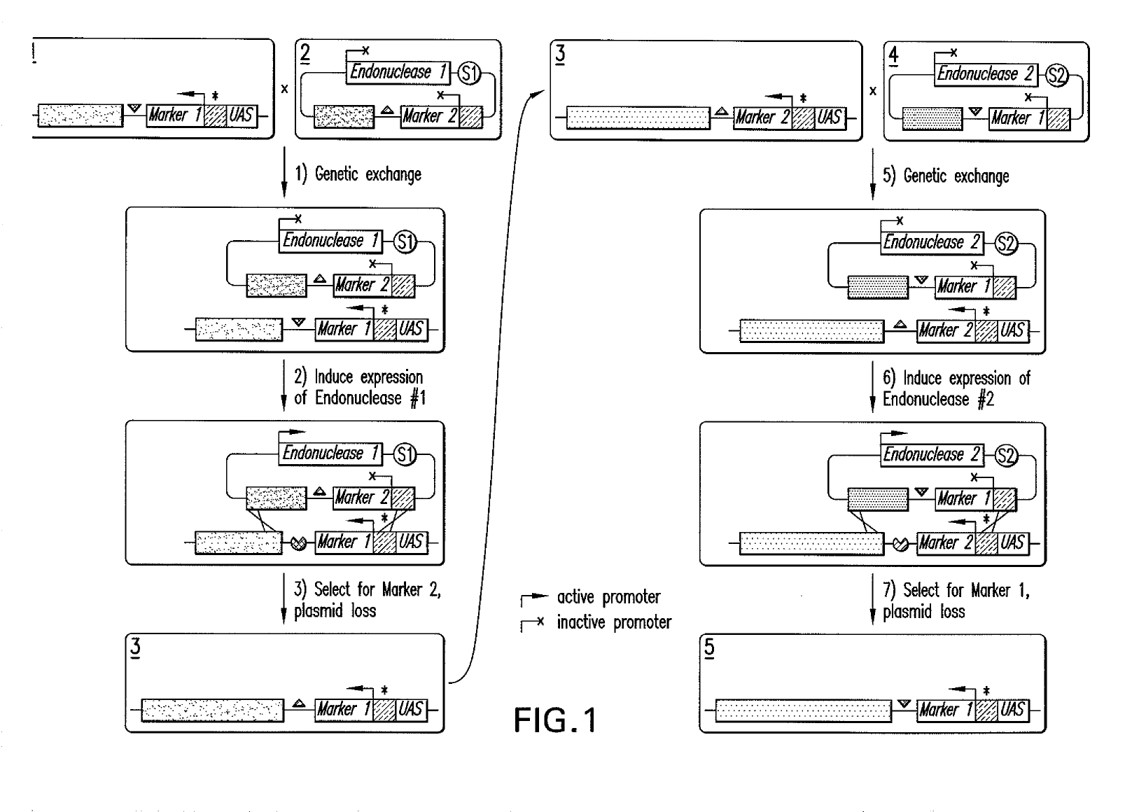

A preferred, non-limiting embodiment of the invention is depicted in FIG. 1. A founder acceptor cell (1) contains a founder acceptor module which preferably is integrated into the host chromosome but which alternatively may be episomal (e.g., carried by a plasmid) comprising (i) a first subfragment of the DNA construct to be assembled (which may be the most downstream region of the construct to be finally produced) downstream of (ii) a cleavage site of a first endonuclease (“Endonuclease 1”; site at downward arrow); (iii) a gene encoding a first selectable marker (“Marker 1”) operably linked to (iv) an active promoter or promoter complex; FIG. 1 depicts the non-limiting embodiment in which a promoter complex is present comprising a promoter (“*”) and an upstream activating sequence (“UAS”) without which the promoter has negligible activity.

A first odd donor cell (2), (which, if the host is yeast, is of opposite mating type relative to the founder acceptor cell), contains a first odd donor complex which may be integrated into the host chromosome or be episomal (the latter being preferred as it facilitates removal) and comprises (i) (optionally) a gene encoding Endonuclease 1 under the control of an inducible promoter; (ii) (optionally) a gene encoding a selectable marker S1 operably linked to a promoter which may be constitutively active or inducible and may or may not be the same promoter that controls expression of Endonuclease 1; and (iii) a first odd donor module comprising (a) a second subfragment of the DNA construct to be assembled, a region of which is homologous to the first subfragment of DNA construct (said homologous region being at least about 30, or at least about 40, or at least about 55, or at least about 70, or at least about 85, or at least about 100 bp in length), downstream of (b) a cleavage site of a second endonuclease (“Endonuclease 2”; site at upward arrow); and (c) a gene encoding a second selectable marker (“Marker 2”) downstream of a region of homology between the first odd donor module and the founder acceptor module and optionally the second even donor module described below (“downstream” means in the direction of transcription). In FIG. 1, this region of homology lies in at least a portion of promoter (“*”), absent its UAS. This homologous region is at least about 30, or at least about 40, or at least about 55, or at least about 70, or at least about 85, or at least about 100 bp in length.

A second (relative to the first (odd) donor cell) even donor cell (4) (which, if the host cell is yeast, is of the opposite mating type as the progeny acceptor cell with which it will undergo genetic exchange), contains a second even donor complex comprising (i) (optionally) a gene encoding Endonuclease 2 under the control of an inducible promoter; (ii) (optionally) a gene encoding a selectable marker S2 (which may or may not be the same as S1) operably linked to a promoter which may be constitutively active or inducible and may or may not be the same promoter that controls expression of Endonuclease 2; and (iii) a second even donor module comprising (a) a third subfragment of the DNA construct to be assembled (solid bar), a region of which is homologous to the second subfragment of DNA construct (said homologous region being at least about 30, or at least about 40, or at least about 55, or at least about 70, or at least about 85, or at least about 100 bp in length), downstream of (b) a cleavage site of Endonuclease 1, downward arrow; and (c) the gene encoding Marker 1 downstream of a region of homology between the second even donor module and the progeny acceptor module and optionally the third odd donor module described below. In FIG. 1, this region of homology lies in at least a portion of promoter (“*”), absent its UAS. This region of homology is at least about 30, or at least about 40, or at least about 55, or at least about 70, or at least about 85, or at least about 100 bp in length.

A third and subsequent odd-number donor cells contain a third or subsequent odd donor complex comprising a third or subsequent odd donor module comprising the same elements as the first odd donor complex and module of the first odd donor cell, except that the subfragment of the DNA construct to be assembled is progressively further upstream and shares a region of homology with the subfragment of DNA construct in the even-number donor cell which is utilized before said odd-number donor cell, said homologous region being at least about 30, or at least about 40, or at least about 55, or at least about 70, or at least about 85, or at least about 100 bp in length.

A fourth and subsequent even-number donor cells contain a fourth or subsequent even donor complex comprising a fourth or subsequent donor module comprising the same elements as the second even donor complex and module of the second even donor cell, except that the subfragment of the DNA construct to be assembled is progressively further upstream and shares a region of homology with the subfragment of DNA construct in the odd-number donor cell which is utilized before said even-number donor cell, said homologous region being at least about 30, or at least about 40, or at least about 55, or at least about 70, or at least about 85, or at least about 100 bp in length.

To produce a DNA construct from X component subfragments of DNA, genetic exchange is allowed to occur between the founder acceptor cell and the first odd donor cell (e.g., where the hosts cells are yeast cells, a haploid founder acceptor cell is mated with a haploid first donor cell to form a diploid cell, or, alternatively, a haploid founder acceptor cell may be transformed with DNA comprising a first odd donor complex comprising a first odd donor module) and a progeny cell expressing Marker 1 (and optionally) S1) is selected. Expression of Endonuclease 1 is then induced. Endonuclease 1 creates a double strand break between the first subfragment of DNA construct to be assembled and the gene encoding Marker 1, promoting homologous recombination between (i) homologous regions of the first and second subfragments of DNA construct and (ii) between regions upstream of Marker genes 1 and 2 (for example, but not by way of limitation, within the promoter regions). A first progeny acceptor cell is then selected for expression of Marker 2, which is now operably linked to the active promoter (in FIG. 1, promoter */UAS). Note that, as a result of homologous recombination, the endonuclease specificity of the cleavage site between the DNA construct assembly and the marker gene has been switched from Endonuclease 1 susceptibility to Endonuclease 2 susceptibility, and the marker gene has been switched from a gene encoding Marker 1 to a gene encoding Marker 2. Preferably, where the donor module is carried episomally (e.g., in a plasmid), said plasmid is removed by counterselection (e.g., selection against expression of S1). In other non-limiting embodiments, where the host cell is yeast and the donor module is carried on the chromosome, the yeast, in diploid form, can be sporulated and those haploids that do not contain the donor module can be selected for further use.

Next, genetic exchange is allowed to occur between the first progeny acceptor cell and the second even donor cell (for example, where the acceptor and donor cells are yeast, the diploid first progeny acceptor cell is sporulated, and a haploid first progeny acceptor cell is mated with a haploid second even donor cell to produce a diploid progeny cell or, alternatively, the first progeny acceptor cell may be transformed with DNA comprising a second even donor complex comprising a second even donor module) and a progeny cell expressing Marker 2 (and optionally S2) is selected. Expression of Endonuclease 2 is then induced. Endonuclease 2 creates a double strand break between the joined first and second subfragments of DNA construct to be assembled and the gene encoding Marker 2, promoting homologous recombination between (i) homologous regions of the second and third subfragments of DNA construct and (ii) between regions upstream of Marker genes 2 and 1 (for example, but not by way of limitation, within the promoter regions). Second progeny acceptor yeast cells are then selected for expression of Marker 1, which is now operably linked to the active promoter (in FIG. 1, promoter */UAS). Note that, as a result of homologous recombination, the endonuclease specificity of the cleavage site between the DNA construct assembly and the marker gene has been switched from Endonuclease 2 susceptibility to Endonuclease 1 susceptibility, and the marker gene has been switched from a gene encoding Marker 2 to a gene encoding Marker 1.

The above process may be repeated any number of times by mating a progeny acceptor cell with a donor cell and, in these matings, alternating between a donor cell having an even donor module to a donor cell having an odd donor module (or, alternatively, by iteratively transforming a progeny acceptor cell with DNA comprising a donor module and alternating between even and odd donor modules)(wherein a donor module may be comprised in a donor complex), wherein successive donor modules contain overlapping successive portions of the DNA to be constructed, until all X subfragments of DNA construct have been assembled. In a related, non-limiting embodiment, the inducible genes encoding endonuclease 1 and/or 2 may be contained in a chromosome rather than carried on a plasmid. For example, but not by way of limitation, an orthogonal inducible promoter, such as a GAL-inducible, tetracycline-inducible, or CUP1 promoter may be used.

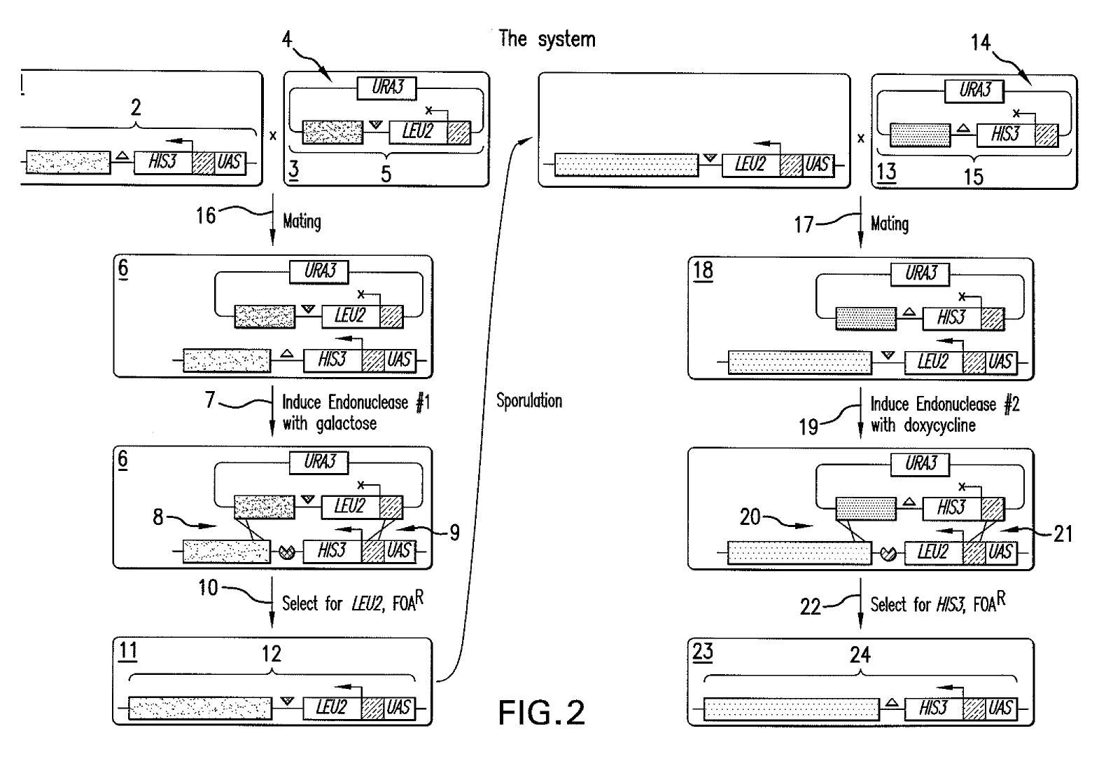

A preferred, non-limiting embodiment of the invention is practiced in yeast using a system as outlined in FIG. 2. As set forth below, genetic exchange in this system may be accomplished either by mating between cells or by transforming an acceptor cell with DNA comprising a donor module.

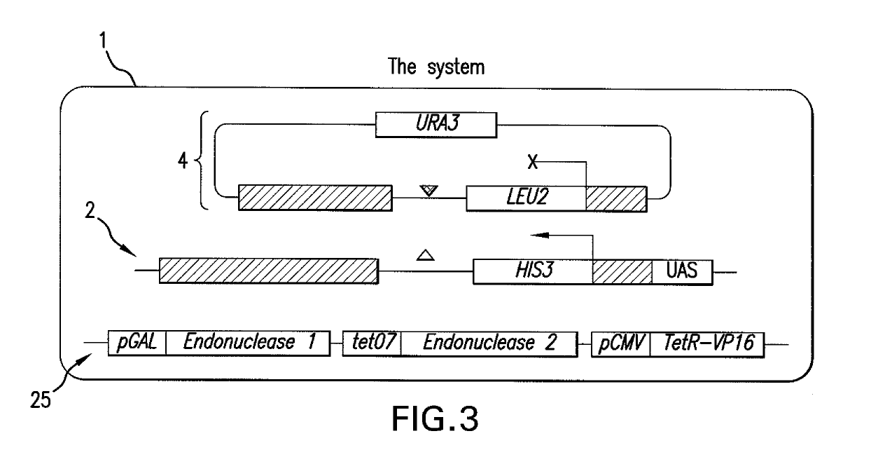

A founder acceptor yeast cell (1) contains an “acceptor module” (2) which preferably is integrated into the yeast chromosome but which alternatively may be episomal (e.g., carried by a plasmid) comprising (i) a first subfragment of the DNA construct to be assembled (which is the most downstream region of the construct to be finally produced) downstream of (ii) a cleavage site of a first endonuclease (“Endonuclease 1”; site at upward open arrow); (iii) a gene encoding a first selectable marker, HIS3 (“Marker 1) operably linked to (iv) a promoter (“*”); and (v) an upstream activating sequence (“UAS”) without which the promoter has negligible activity. For example, but not by way of limitation, endonuclease 1 may be HO endonuclease and endonuclease 2 may be SceI endonuclease; alternatively, endonuclease 1 may be SceI endonuclease and endonuclease 2 may be HO endonuclease; or endonuclease 1 may be HO endonuclease and endonuclease 2 may be the DmoI variant; or endonuclease 1 may be the DmoI variant and endonuclease 2 may be HO endonuclease; or endonuclease 1 may be SceI endonuclease and endonuclease 2 may be the DmoI variant; or endonuclease 1 may be the DmoI variant and endonuclease 2 may be SceI endonuclease. For example, but not by way of limitation, the promoter complex may comprise a promoter and UAS elements. According to this specific embodiment, and not by way of limitation, the founder acceptor cell comprises (as chromosomal DNA) a nucleic acid comprising a nucleic acid encoding Endonuclease 1 operably linked to the pGAL promoter; a nucleic acid comprising a nucleic acid encoding Endonuclease 2 operably linked to the tet07 promoter; and a nucleic acid comprising a nucleic acid encoding TetR′-VP16 operably linked to the pCMV immediate early promoter (Gari et al., 1997, Yeast 13: 837, source, EUROSCARF). FIG. 3 illustrates a non-limiting embodiment of the invention showing (25) a region of chromosomal DNA comprising the genes encoding Endonucleases 1 and 2; a region of chromosomal DNA comprising an acceptor module (2) and the first odd donor complex (4) from FIG. 2.

A first odd donor yeast cell (3), of opposite mating type relative to the founder acceptor yeast cell contains an extrachromosomal first odd donor complex comprising the selectable marker URA3 operably linked to a promoter element such as its endogenous promoter and an odd donor module comprising (i) a second subfragment of the DNA construct to be assembled, a region of which is homologous to the first subfragment of DNA construct (said homologous region being at least about 30, or at least about 40, or at least about 55, or at least about 70, or at least about 85, or at least about 100 bp in length), downstream of (ii) a cleavage site of a second endonuclease (“Endonuclease 2”; site at downward arrow); and (iii) a gene encoding a second selectable marker, LEU2, operably linked to (iv) a nucleic acid sequence homologous to the promoter of the acceptor module (preferably the same promoter) absent its UAS. This region of homology is at least about 30, or at least about 40, or at least about 55, or at least about 70, or at least about 85, or at least about 100 bp in length.

A second even donor yeast cell (4), of the same mating type relative to the founder acceptor yeast cell, contains a second even donor complex comprising a gene encoding selectable marker URA3 operably linked to a promoter element such as its endogenous promoter and an even donor module comprising (i) a third subfragment of the DNA construct to be assembled, a region of which is homologous to the second subfragment of DNA construct (said homologous region being at least about 30, or at least about 40, or at least about 55, or at least about 70, or at least about 85, or at least about 100 bp in length), downstream of (ii) a cleavage site of Endonuclease 1, upward open arrow; and (iv) the gene encoding HIS3 operably linked to (iv) a nucleic acid sequence homologous to the promoter of the acceptor module (preferably the same promoter) absent its UAS. This region of homology is at least about 30, or at least about 40, or at least about 55, or at least about 70, or at least about 85, or at least about 100 bp in length.

A third and subsequent odd-number donor yeast cells have the same mating type and contain an odd donor module comprising the same elements as the odd donor module of the first odd donor cell, except that the subfragment of the DNA construct to be assembled is progressively further upstream and shares a region of homology with the subfragment of DNA construct in the even-number donor cell which is utilized before said odd-number donor cell, said homologous region being at least about 30, or at least about 40, or at least about 55, or at least about 70, or at least about 85, or at least about 100 bp in length.

A fourth and subsequent even-number donor yeast cells have the same mating type and contain an even donor module comprising the same elements as the even donor module of the second even donor cell, except that the subfragment of the DNA construct to be assembled is progressively further upstream and shares a region of homology with the subfragment of DNA construct in the odd-number donor cell which is utilized before said even-number donor cell, said homologous region being at least about 30, or at least about 40, or at least about 55, or at least about 70, or at least about 85, or at least about 100 bp in length.

To produce a DNA construct from X component subfragments of DNA, a haploid founder acceptor cell is mated with a haploid first donor cell (or the founder acceptor cell is transformed with DNA comprising a first odd donor complex), and diploid cells expressing HIS3 and URA3 are selected. Expression of Endonuclease 1 is then induced by adding galactose to the cell culture medium. Endonuclease 1 creates a double strand break between the first subfragment of DNA construct to be assembled and the gene encoding HIS3, promoting homologous recombination between (i) homologous regions of the first and second subfragments of DNA construct and (ii) between regions upstream of HIS3 and LEU2 (as shown by FIG. 2, not by way of limitation, within the promoter regions). First progeny acceptor yeast cells are then selected for expression of LEU2, which is now operably linked to the active promoter/UAS complex. Note that, as a result of homologous recombination, the endonuclease specificity of the cleavage site between the DNA construct assembly and the marker gene has been switched from Endonuclease 1 susceptibility to Endonuclease 2 susceptibility, and the marker gene has been switched from a gene encoding HIS3 to a gene encoding LEU2. Preferably, where the odd donor module is carried episomally (e.g., in a plasmid), said plasmid is removed by counterselection (e.g., selection against expression of URA3).

Next, the diploid first progeny acceptor cell is sporulated, and a haploid first progeny acceptor cell is mated with a haploid second donor cell (or the first progeny acceptor cell is transformed with DNA comprising a second even donor complex), and diploid cells expressing LEU2 and URA3 are selected. Expression of Endonuclease 2 is then induced by adding doxycycline to the culture medium. Endonuclease 2 creates a double strand break between the joined first and second subfragments of DNA construct to be assembled and the gene encoding LEU2, promoting homologous recombination between (i) homologous regions of the second and third subfragments of DNA construct and (ii) between regions upstream of LEU2 and HIS3 (as shown by FIG. 2, not by way of limitation, within the promoter regions). Second progeny acceptor yeast cells are then selected for expression of HIS3, which is now operably linked to the active promoter/UAS complex. Note that, as a result of homologous recombination, the endonuclease specificity of the cleavage site between the DNA construct assembly and the marker gene has been switched from Endonuclease 2 susceptibility to Endonuclease 1 susceptibility, and the marker gene has been switched from a gene encoding LEU2 to a gene encoding HIS3.

The above process may be repeated any number of times by mating a progeny acceptor cell with a donor cell and, in these matings, alternating between a donor cell having an even donor module to a donor cell having an odd donor module (or, alternatively, by iteratively transforming a progeny acceptor cell with DNA comprising a donor module and alternating between even and odd donor modules)(wherein a donor module may be comprised in a donor complex), wherein successive donor modules contain overlapping successive portions of the DNA to be constructed, until all X subfragments of DNA construct have been assembled.

In a related, non-limiting embodiment to the system shown in FIGS. 2 and 3, the inducible genes encoding Endonuclease 1 and/or 2 may be contained in a plasmid. Alternatively, genes encoding Endonucleases 1 and 2, each under the control of an inducible promoter, may be separately placed in odd and even donor complexes, so that if a donor complex carries the gene encoding Endonuclease 1, then the donor module in that complex contains a cleavage site for Endonuclease 2, and vice-versa. For example, but not by way of limitation, an orthogonal inducible promoter, such as a GAL-inducible, tetracycline-inducible, or CUP1 promoter may be used (including nucleic acid encoding any other relevant component, such as, in the case of tet07, nucleic acid encoding TetR′-VP16 operably linked to a suitable promoter, may be used).

### 5.6 Kits

The present invention provides for a kit comprising (i) a nucleic acid which may be used to produce an acceptor module (“an acceptor cassette”) and (ii) a nucleic acid which may be used to produce an odd donor module (“an odd donor cassette”), and preferably (iii) a nucleic acid which may be used to produce an even donor module (“an even donor cassette”). Where element (iii) is absent, an even donor module may be produced using nucleic acid elements (i) and (ii) using standard recombinant DNA technology.

In particular non-limiting embodiments of the invention, the acceptor cassette comprises (i) a restriction site for inserting a DNA subfragment of the complex to be assembled; (ii) an endonuclease cleavage site; and (iii) a gene encoding a first selectable marker operably linked to (iv) an active promoter or promoter complex. The endonuclease cleavage site, gene encoding a selectable marker, and active promoter or promoter complex elements are described in the “ACCEPTOR MODULE” section, above. Other alternative methods for inserting a construct known to those skilled in the art may also be used.

In particular non-limiting embodiments of the invention, the odd donor cassette comprises (i) a restriction site for inserting a DNA subfragment of the complex to be assembled, (ii) an endonuclease cleavage site; (iii) a gene encoding a second selectable marker which differs from the gene for a first selectable marker in the acceptor module; and (iv) a region upstream of the gene of (iii) which is homologous to a region of the acceptor cassette (or the even donor cassette, if present) such that homologous recombination between the corresponding regions in the corresponding odd donor module and acceptor modules would place the gene encoding the second selectable marker under transcriptional control of the active promoter or promoter complex and would inactivate the gene encoding the first selectable marker.

In a preferred embodiment, the endonuclease cleavage site of the odd donor cassette is different from the endonuclease cleavage site of the acceptor cassette and these two sites are cleaved by different endonucleases.

The odd donor cassette may optionally further comprise, outside of the region designed to become the donor module, a nucleic acid encoding an endonuclease that cleaves at the endonuclease cleavage site of the acceptor cassette, said nucleic acid operably linked to a promoter which is preferably an inducible promoter.

The odd donor cassette may further optionally comprise, also outside of the region designed to become the donor module, a nucleic acid encoding a selectable marker different from the selectable markers that are to be present in the acceptor module and donor module(s), said nucleic acid operably linked to a promoter that is optionally inducible.

In this description of the odd donor cassette, the endonuclease cleavage site, gene encoding a selectable marker of the donor module or donor complex, nucleic acid encoding the endonuclease, promoters and inducible promoters, are as described in the “DONOR MODULE” section, above. Other alternative methods for inserting a construct known to those skilled in the art may also be used.

In particular non-limiting embodiments of the invention, an even donor cassette comprises (i) a restriction site for inserting a DNA subfragment of the complex to be assembled, (ii) the same endonuclease cleavage site which is present in the acceptor cassette; (iii) a gene encoding the same selectable marker present in the acceptor cassette (the first selectable marker); and (iv) a region upstream of the gene of (iii) which is homologous to a region of the odd donor cassette (or the acceptor cassette) such that homologous recombination between the corresponding regions in the corresponding even donor module and proogeny acceptor module would place the gene encoding the first selectable marker under transcriptional control of the active promoter or promoter complex and would inactivate the gene encoding the second selectable marker.

In a preferred embodiment, the endonuclease cleavage site of the even donor cassette is different from the endonuclease cleavage site of the odd donor cassette and these two sites are cleaved by different endonucleases, but the endonuclease sites of the even donor cassette and the acceptor cassette are the same and are cleaved by the same endonuclease.

The even donor cassette may further optionally comprise, outside of the region designed to become the donor module, a nucleic acid encoding an endonuclease that cleaves at the endonuclease cleavage site of the acceptor cassette, said nucleic acid operably linked to a promoter which is preferably an inducible promoter.

The even donor cassette may further optionally comprise, also outside of the region designed to become the donor module, a nucleic acid encoding a selectable marker different from the selectable markers that are to be present in the acceptor module and donor module(s), said nucleic acid operably linked to a promoter that is optionally inducible.

In this description of the even donor cassette, the endonuclease cleavage site, gene encoding a selectable marker of the donor module or donor complex, nucleic acid encoding the endonuclease, promoters and inducible promoters, are as described in the “DONOR MODULE” section, above. Other alternative methods for inserting a construct known to those skilled in the art may also be used.

A kit may optionally further comprise nucleic acids comprising nucleic acids encoding one or two endonucleases, operably linked to an inducible promoter(s), for use according to the invention. In a non-limiting embodiment, the kit may optionally contain a nucleic acid construct comprising a sequence encoding a first endonuclease operably linked to a first inducible promoter and optionally a sequence encoding a second endonuclease operably linked to the same or a different inducible promoter. Alternatively, a sequence encoding a second endonuclease operably linked to the same or a different inducible promoter may be comprised in a separate nucleic acid. Said construct(s) may further comprise sequences flanking the endonuclease gene-promoter, said flanking sequences being homologous to nucleic acid sequence in the intended host cell, to facilitate integration into the host genome. Alternatively or in addition, said construct(s) may comprise sequences flanking the endonuclease gene-promoter, said flanking sequences being homologous to nucleic acid sequence in a non-chromosomal DNA (e.g., a donor complex, a plasmid or non-plasmid vector), to facilitate integration into said non-chromosomal DNA.

In non-limiting embodiments of the invention, the kit may further comprise one or more primer which may be used to generate DNA subfragments to be assembled into the construct, for example by PCR. Said primer may comprise a sequence that can facilitate cloning into the restriction site of the acceptor and/or donor cassette(s).

In non-limiting embodiments, the kit may further comprise a restriction enzyme and/or a polymerase enzyme and/or a ligase that may be used in preparation of the acceptor or donor module(s).

In non-limiting embodiments, the kit may further comprise a selection agent that selects for or counterselects against a selection marker, for example an antibiotic or metabolic agent.

Other elements that may optionally be included in a kit according to the invention include an acceptor cell line that contains the acceptor cassette, and optionally a correlative donor cell line comprising a donor cassette.

### 5.7 Convergent Reiterative Recombination

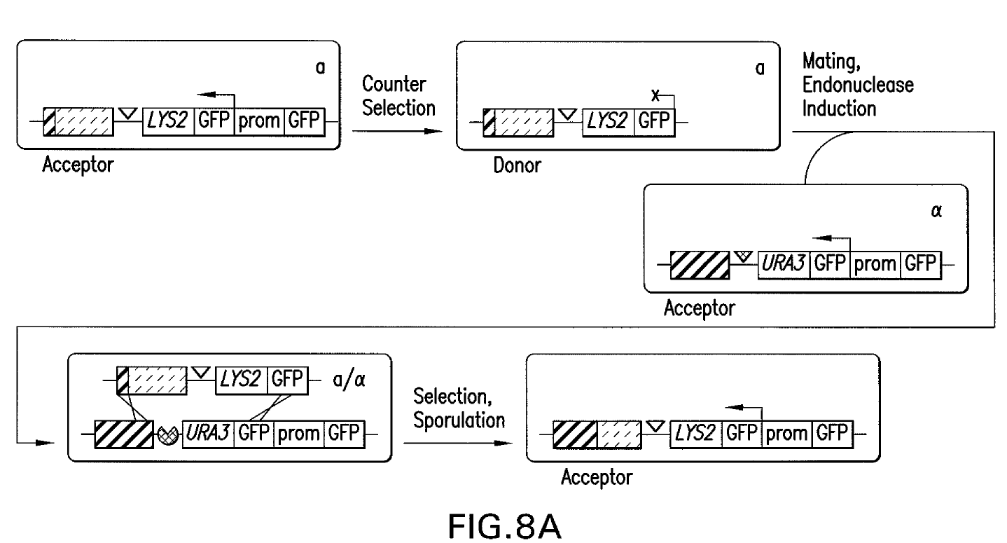

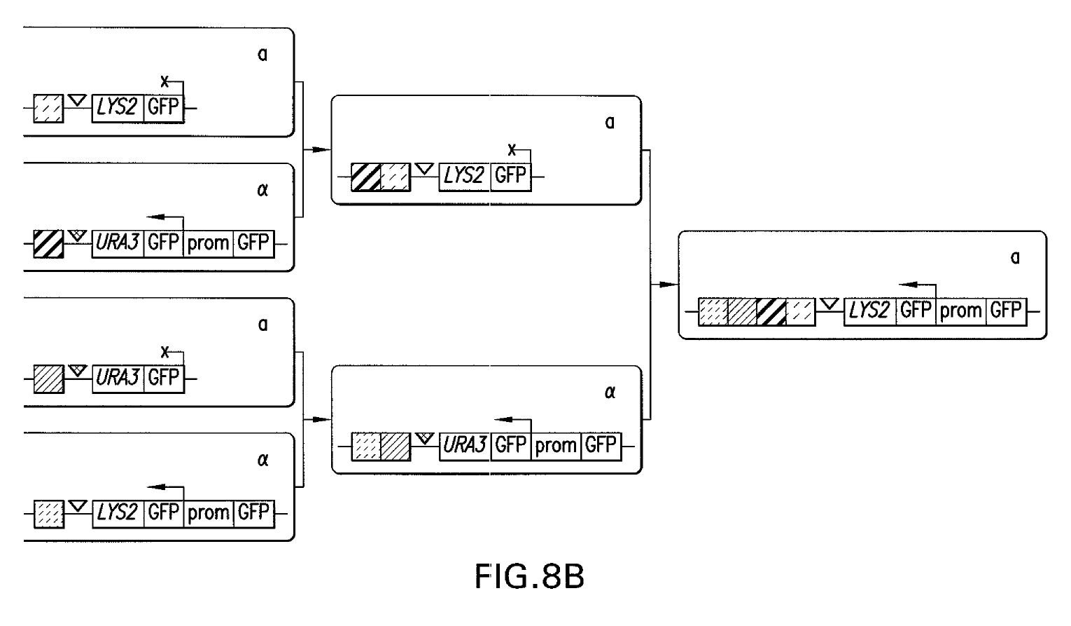

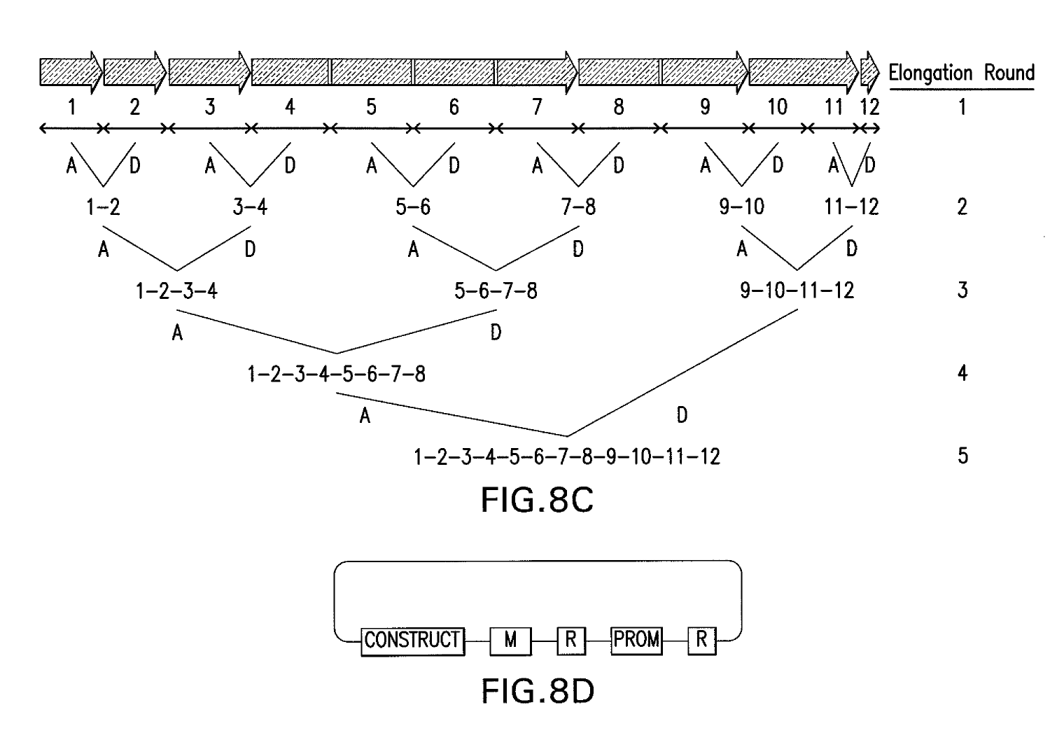

By analogy to convergent organic syntheses, the present invention provides for the use of reiterative recombination to assemble DNA constructs in a convergent rather than a linear fashion. Convergent reiterative recombination utilizes genetic exchange (for example, but not by way of limitation, sexual reproduction), and, while it shares many elements with linear reiterative recombination (described above), has a modification in the acceptor module which allows it to become a donor module. This modification results in the excisability of the promoter element which drives expression of a selectable marker. FIG. 8D presents a schematic depiction of an acceptor module for use in convergent reiterative recombination, which comprises a portion of the DNA construct to be assembled, and, upstream of the construct, a selectable marker (“M”) operatively linked to a promoter element (“PROM”), where the promoter element is flanked by direct repeat elements (“R”). The direct repeats may be non-coding sequenes or may encode a protein product. A recombination event between the direct repeats results in excision of the promoter element. This recombination event may be detected by the loss of expression of the selectable marker, e.g. by counterselection. Genetic exchange, e.g. mating and sporulation where the organism is yeast, may then be used to bring donor and acceptor modules together.

Accordingly, the present invention provides for combining a first DNA construct with a second DNA construct by convergent reiterative recombination by a method comprising:

(A) preparing the first DNA construct by a method comprising:
(Ai) providing a first acceptor cell containing a convergent acceptor module comprising, downstream to upstream (a) a first DNA subfragment that is to be assembled to form the construct; (b) an endonuclease cleavage site; and (c) a gene encoding a first selectable marker operably linked to (d) an active promoter or active promoter complex, wherein the active promoter or promoter complex is flanked by direct repeats;
(Aii) providing a first donor cell containing a first donor module comprising, downstream to upstream, (a) a second DNA subfragment, that is to be joined to the first DNA subfragment of (i) and that shares a region of homology with it; (b) an endonuclease cleavage site; (c) a gene encoding a second selectable marker which is not the same as the first selectable marker; and (d) a region upstream of the gene of (c) which is homologous to a region of the convergent acceptor module such that recombination between the convergent acceptor module and the first donor module in this region places the gene encoding the second selectable marker under transcriptional control of the active promoter or active promoter complex and inactivates the gene encoding the first selectable marker; and
(Aiii) allowing for genetic exchange between the first donor cell and the first acceptor cell;
(Aiv) selecting for expression of the second selectable marker; and then, after culturing under conditions that allow homologous recombination,

(Av) selecting for lack of expression of the second selectable marker (i.e., counterselecting), which indicates that the promoter element has been excised, such that the module contained by the a cell selected in this subparagraph has been transformed into a (third) donor module comprising the first DNA construct and said cell is a third donor cell;

(B) preparing the second DNA construct by a method comprising:
(Bi) providing a second acceptor cell containing a second acceptor module comprising, downstream to upstream (a) a third DNA subfragment that is to be assembled to form the construct; (b) an endonuclease cleavage site; and (c) a gene encoding a third selectable marker (which may or may not be the same as the first or second selectable markers) operably linked to (d) an active promoter or active promoter complex;
(Bii) providing a second donor cell containing a second donor module comprising, downstream to upstream, (a) a fourth DNA subfragment, that is to be joined to the third DNA subfragment of (i) and that shares a region of homology with it; (b) an endonuclease cleavage site; (c) a gene encoding a fourth selectable marker which is not the same as the third selectable marker; and (d) a region upstream of the gene of (c) which is homologous to a region of the acceptor module such that recombination between the acceptor module and the second donor module in this region places the gene encoding the fourth selectable marker under transcriptional control of the active promoter or active promoter complex and inactivates the gene encoding the third selectable marker; and
(Biii) allowing for genetic exchange between the second donor cell and the second acceptor cell;
(Biv) selecting for expression of the fourth selectable marker, wherein the module in a cell expressing the fourth selectable marker is a third acceptor module comprising the second DNA construct and said cell is a third acceptor cell; and

(C) allowing for genetic exchange between the third acceptor cell of (Biv) and the third donor cell of A(v); and

(D) selecting for the second selectable marker, wherein the module in a cell expressing the second selectable marker comprises the second DNA construct joined to the first DNA construct.

### 5.8 Prophetic Example 1

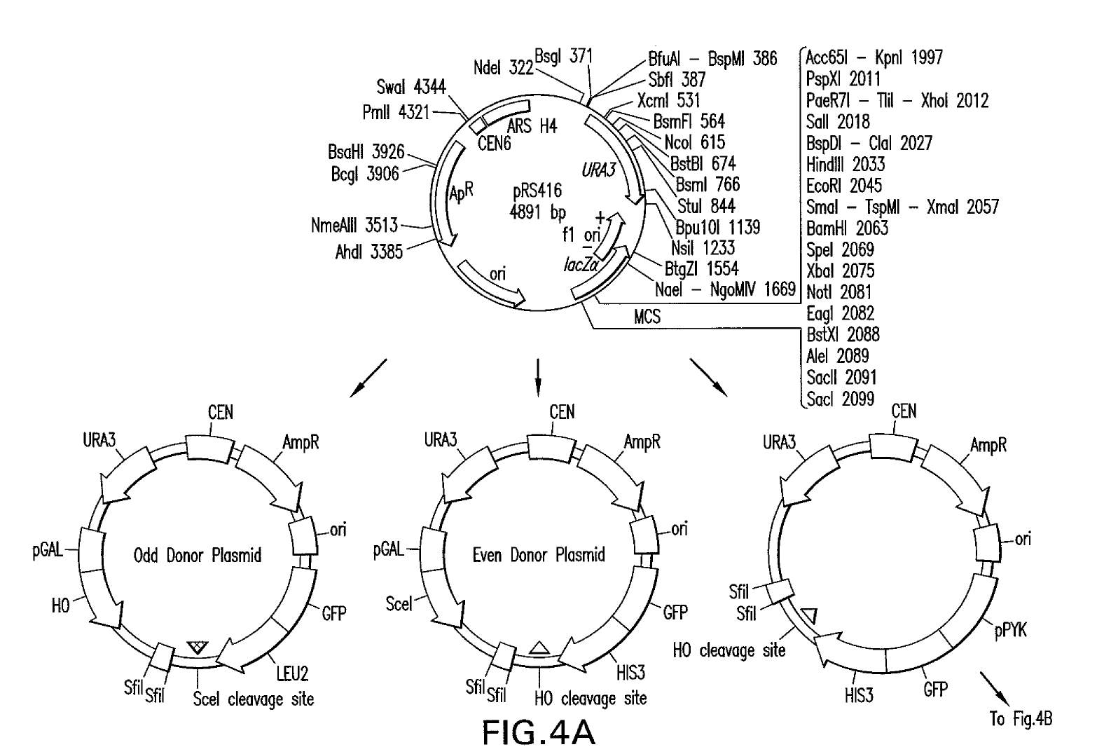

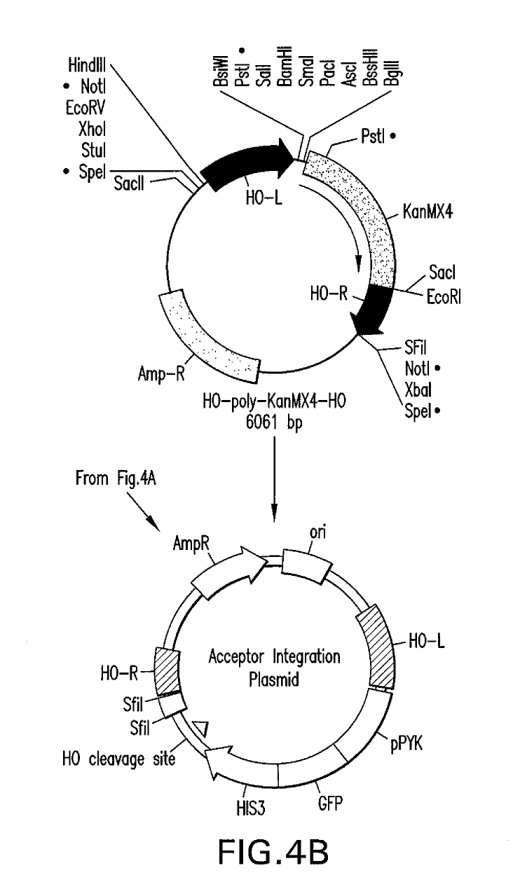

FIG. 4 presents diagrams that illustrate construction of odd and even donor complexes providing odd and even donor modules from a single parent plasmid, pRS416; (Acc. No. UO3450, yeastgenome.org, submitted 11 Nov. 1993 by David J. Stillman, Dept. of Cellular, Viral and Molecular Biology, University of Utah Medical Center, Salt LakeCity, Utah 84132 USA; Sikorski and Hieter, 1989, Genetics 122:19-27; Christianson, et al., 1992, Gene 110; 119-122) which is a shuttle vector comprising yeast CEN6 sequence, the URA3 gene, the ampicillin resistance gene, an origin of replication, and a multiple cloning site.

To produce odd/even donor cassettes, pRS416 may be cleaved in its multiple cloning site, and a construct may be inserted between these sites comprising a marker gene (i) upstream of which is a sequence having a region of homology with the corresponding acceptor module and optionally the next donor module to be used and (ii) downstream of which is an endonuclease cleavage site, as described above. For example, the construct may comprise an upstream homology region (as described in the preceding sentence), a selection marker gene which is a fusion between green fluorescent protein (“GFP”) and HIS3 and an endonuclease cleavage site which is TTTCAGCTTTCCGCAACAGTATAA (SEQ ID NO: 11), recognized by HO endonuclease. Optionally, a construct encoding an inducible endonuclease gene and a stuffer region to facilitate insertion of DNA subfragments may also be inserted in the multiple cloning site. To produce a donor cassette of the opposite polarity (i.e., even as opposed to odd, odd as opposed to even), a second construct may be inserted at the same site in another cleaved pRS416 molecule comprising an upstream homology region, a selection marker gene which is a fusion between LEU2 and GFP, and an endonuclease cleavage site which may be the same but preferably is different from the endonuclease cleavage site paired with HIS3 in the first construct. Optionally, a construct encoding an inducible endonuclease gene and a stuffer region to facilitate insertion of DNA subfragments may also be inserted in the multiple cloning site.

Once odd and even donor cassettes have been prepared, subfragments of the DNA construct may be introduced such that overlapping fragments are sequentially placed in alternating odd and even donor cassettes. A subfragment of the DNA construct to be assembled may be inserted between SfiI sites in donor cassette created in the pRS416 vector. For a odd/even donor pair, subfragments that share at least a 40 bp homologous region may be prepared by producing an at least 40 bp oligonucleotide primer corresponding to the upstream end of the first subfragment to be joined, of which the 5′→3′ strand serves as one member of a primer pair to synthesize the first subfragment to be assembled (together with a second 3′←5′ oligonucleotide primer bordering the downstream end of the construct and comprising a SfiI cleavage site) and the 5′→3′ strand serves as one member of a primer pair to synthesize the second subfragment to be assembled (together with a third 5←3′ primer lying at the upstream end of the second subfragment to be joined, the 5′→3′ strand of which serves as the downstream primer for the third subfragment to be joined, and so on until the most upstream subfragment of the construct is reached and a SfiI cleavage site is added).

To produce an acceptor cassette, pRS416 may be cleaved in its multiple cloning site, and a construct may be inserted between these sites comprising a marker gene (i) upstream of which is an active promoter or promoter complex, (ii) upstream of which is a sequence having a region of homology with the corresponding acceptor module and optionally the next donor module to be used and (iii) downstream of which is an endonuclease cleavage site. Optionally, a construct comprising a stuffer region to facilitate insertion of DNA subfragments may also be inserted in the multiple cloning site. This acceptor cassette may be cloned into the HO-poly-KanMX4-HO plasmid, which has been cleaved with EcoRI and BglII to remove the KanMX marker, to produce an acceptor integration plasmid. The acceptor integration plasmid may be used to integrate the acceptor construct into the endogenous locus of the BY4733 MATa-inc derivative to produce the acceptor cell.

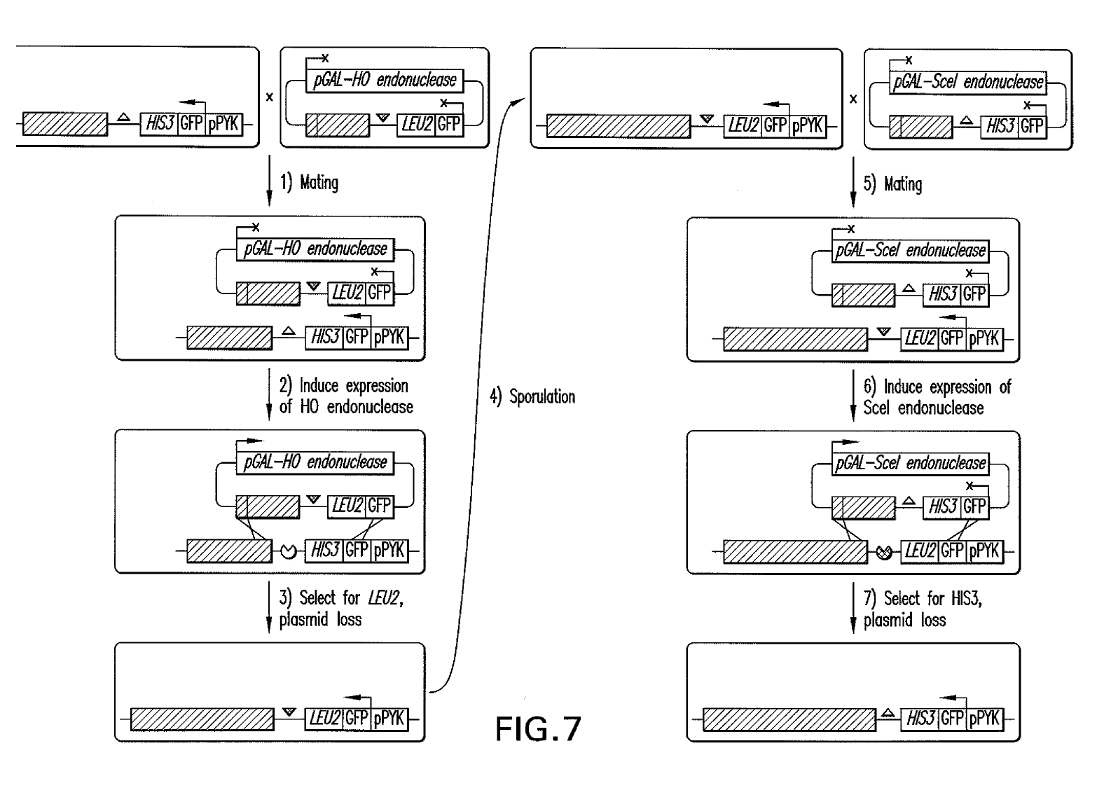

Use of the odd and even donor plasmids and the acceptor cassette/module depicted in FIG. 4 is schematically presented in FIG. 7, and is a non-limiting embodiment of the invention.

### 5.9 Prophetic Example II

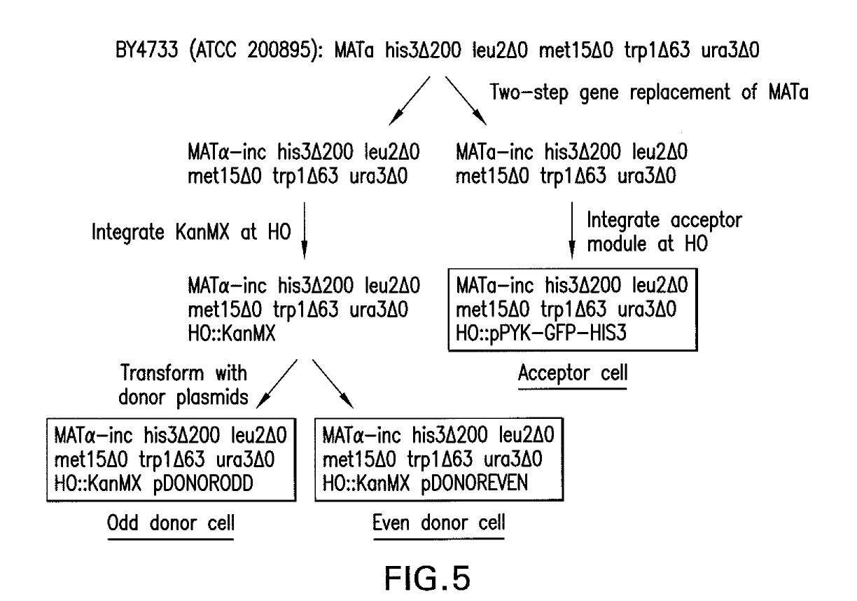

FIG. 5 presents a flow diagram of strain construction. The MATa locus of the yeast strain BY4733 (Source: ATCC; Brachmann, et al. 1998, Yeast 14: 115) may be replaced by MATa-inc and MATα-inc alleles, which cannot be cleaved by HO endonuclease (Weiffenbach, et al. 1983, Proc. Natl. Acad. Sci. USA 80: 3401), via two-step gene replacement (R. Rothstein. Meth. Enzymol. (1991), 194, 284). The MATα-inc strain may have its endogenous HO endonuclease gene replaced with the KanMX marker using the HO-poly-KanMX4-HO integration vector (Ace. No. AF324728; Voth, R. W., et al. 2001, Nucl. Acids Res. 12: e59). The resulting strain may be transformed with odd or even donor plasmids to produce odd or even donor cells, respectively.

### 5.10 Prophetic Example III

See FIG. 8A-D. Convergent synthesis may be used to reconstruct a biosynthetic pathway. Conversion of acceptor modules into donor modules may be accomplished by adding a second copy of GFP upstream of the acceptor module's promoter, creating a direct repeat. URA3 and LYS2, which have both positive and negative selections (Boeke et al., 1984, Molecular & General Genetics 197(2): 345-346; Chattoo et al., 1979, Genetics 93(1): 51-65), may be used as the GFP-marker fusions. Counter selection against these markers may be used to identify cells in which recombination between the GFP repeats has led to deletion of the promoter (Yuan et al., 1990, Genetics 124(2): 263-273), effectively converting the acceptor module into a chromosomal donor module. At all other times, selection for expression of the GFP-marker will eliminate cells that excise the promoter. Haploid cells of opposite mating type (a and α) will be mated to generate diploids with both acceptor and donor modules. Since transformation of donor plasmids will be unnecessary, the two endonucleases will be placed under different inducible promoters (e.g., pGAL1, pCUP1, tetracycline-inducible system (Belli et al., 1998, Nucleic Acids Research 26(4): 942-947; Romanos et al., 1992, Yeast 8(6): 423-488) and integrated into the chromosome. Induction of the appropriate endonuclease followed by selection for marker conversion in the acceptor module should generate the correct recombinants. Sporulation will return cells to the haploid state; the acceptor module's marker may be used to identify cells that received the chromosome with the assembled construct. The acceptor cell may then be taken directly into the next round of elongation or converted into a donor cell via counter selection. Note that both markers and both mating types can be used as both donors and acceptors. FIG. 8C depicts a plan for the convergent construction of the epothilone gene cluster. Twelve round 1 constructs, ranging in size from 1 to 6 kb, will be inserted into acceptor modules using donor plasmids; in subsequent rounds, the convergent assembly strategy will be used

### 6. EXAMPLE I

### Sequential Incorporation of lacZ, gusA, and MET15 Loci into Yeast

The reiterative recombination system used in this example employed, as orthogonal endonucleases, HO and SceI. HO cleaves the MAT locus to stimulate mating-type switching (Strathern et al., 1982, Cell 31(1): 183-192; Haber et al., 1998, Annual Review of Genetics 32: 561-599). SceI is a mitochondrial enzyme involved in rRNA processing and has no recognition sites in yeast nuclear DNA (Colleaux et al., 1988, Proc. Natl. Acad. Sci. U.S.A. 85(16); 6022-6026; Plessis et al., Genetics 130(3): 451-460). These enzymes were placed under the GAL1 promoter, the most widely used inducible promoter in yeast genetics. HIS3 and LEU2 were used as alternating markers; these genes complement the histidine and leucine auxotrophies of many common yeast strains. To provide an upstream homology region, N-terminal GFP fusions of both markers were constructed with an HO or SceI recognition site inserted downstream of their terminators. The GFP-HIS3 construct was placed under a constitutive PYK1 promoter to create an actively expressed acceptor module marker, and both GFP-marker fusions were inserted into centromeric (low-copy) shuttle vectors without promoters to create donor modules. The donor plasmids also each contained a positive and negative selectable URA3 marker, allowing cells to be cured of donor plasmids after each elongation round by growth on 5-fluoroorotic acid (FDA).

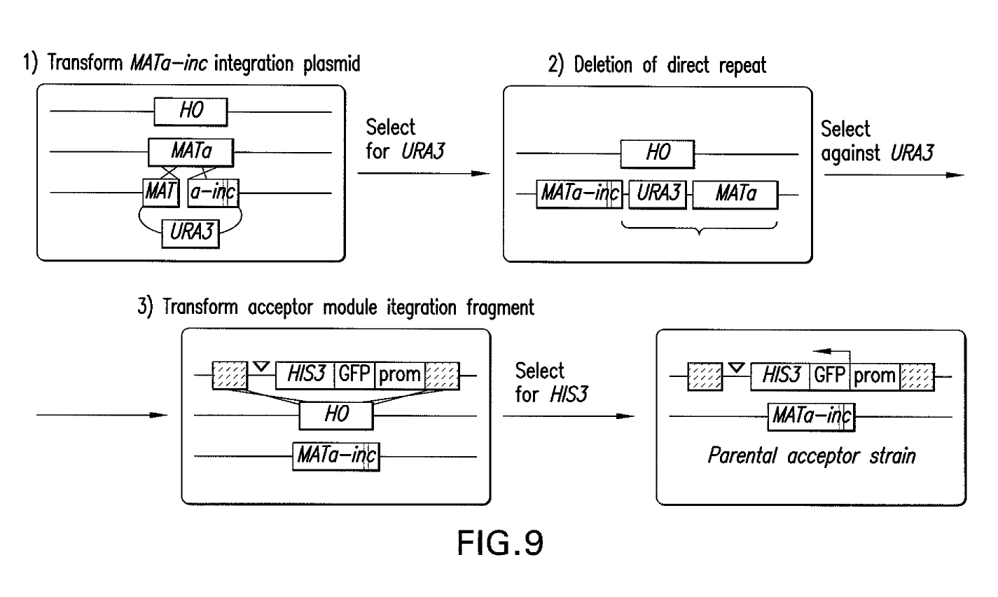

BY4733, a yeast strain having full deletions of all markers discussed in the preceding paragraph (Brachmann et al., 1988, Yeast. 14(2): 115-132), was used as the host strain, thereby eliminating the potential for unwanted homologous recombination events. “Pop-in/pop-out” gene replacement (Scherer et al., 1979, Proc. Natl. Acad. Sci. U.S.A. 76(10): 4951-4955) was used to put a silent mutation in the MAT allele to eliminate its HO recognition site (FIG. 9; Weiffenbach et al., 1983, Proc. Natl. Acad. Sci. U.S.A. 80(11): 3401-3405). Then the acceptor module was inserted into an integration vector targeting the HO locus (Voth et al., 2001, Nucleic Acids Res 29(12): E59-9), simultaneously integrating the construct and eliminating homology to the endonuclease gene in the donor plasmid. This basic parental acceptor strain can be used for the assembly of any desired DNA construct. For some applications, it may be desirable to use a different background strain.

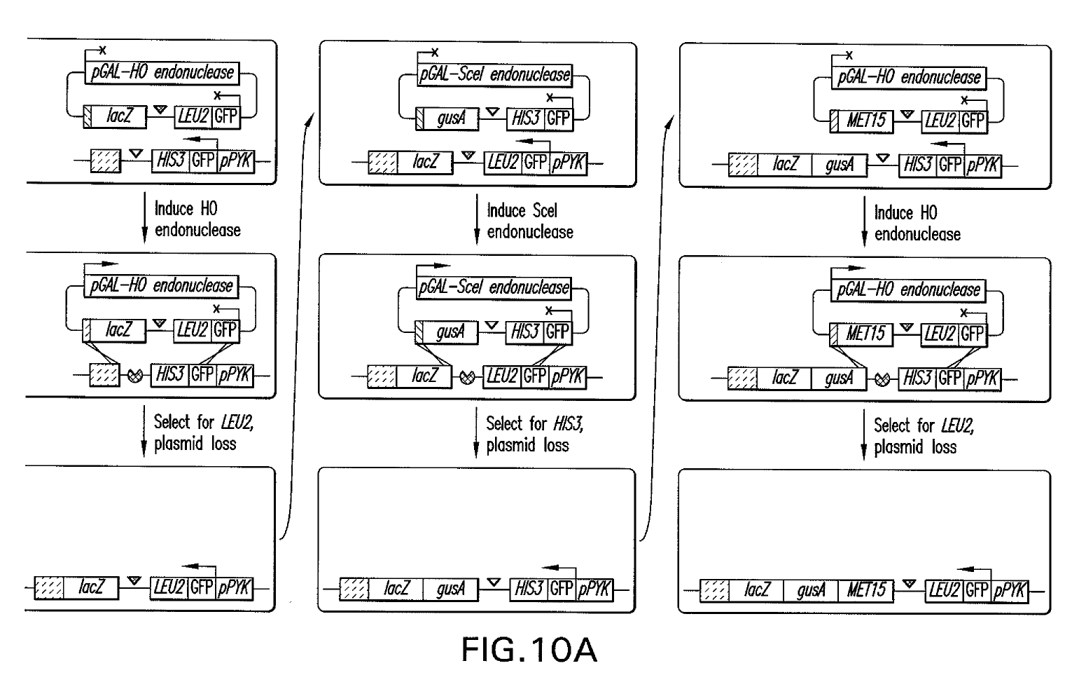

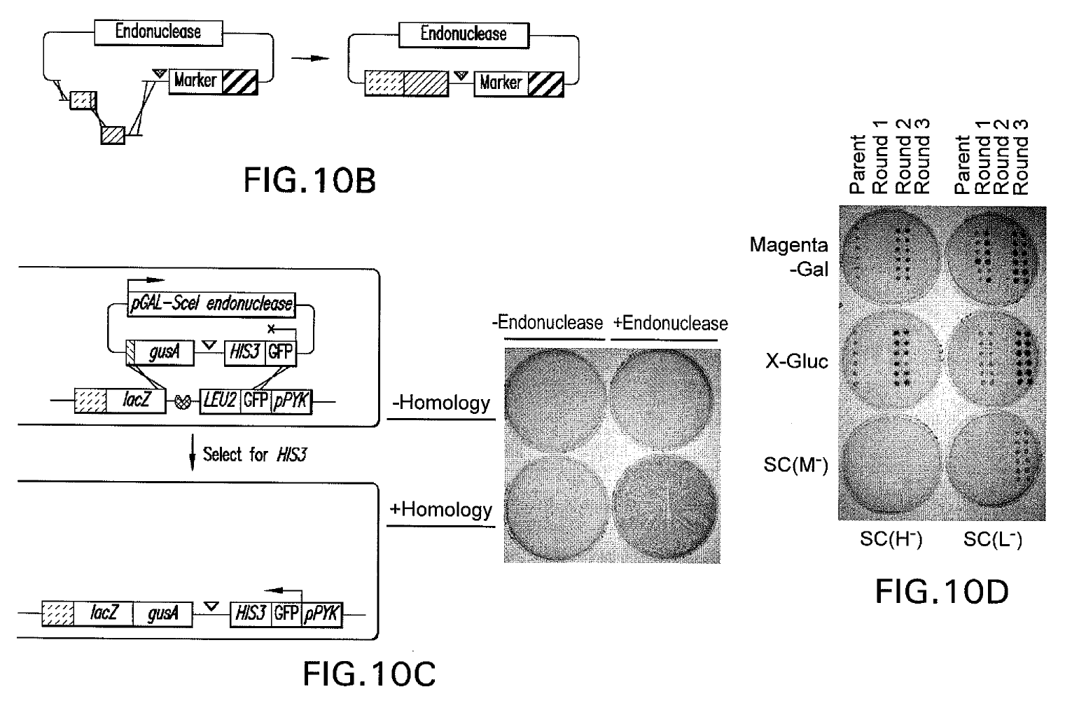

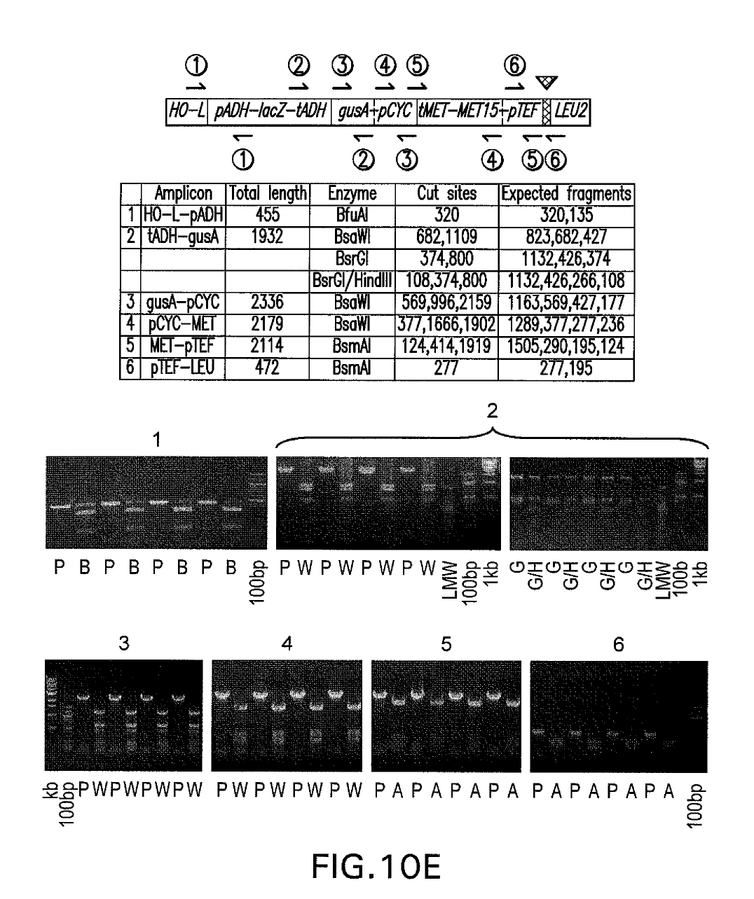

Using the above system, lacZ (β-galactosidase), gusA (β-glucuronidase), and MET15 (which complements methionine auxotrophy), were sequentially integrated into the yeast genomic DNA using three rounds of assembly, creating an 8.5-kb construct (FIG. 10A). Subfragments for integration were PCR amplified as one or two overlapping pieces using primers that incorporated homology 1) to the pieces that are adjacent in the fully assembled construct and 2) to the donor plasmid. PCR products were co-transformed with the digested donor plasmid into the acceptor strain to generate intact donor plasmids by plasmid gap repair (FIG. 10B; Ma et al., 1987, Gene 58(2-1): 201-216), eliminating the need to perform any subcloning.

Using a basic yeast electroporation protocol, as many as 10⁶-10⁸ transformants per transformation were obtained using this technique (Peralta-Yahya et al., 2008, J Am Chem Soc 130(51): 17446-52 (NIHMS91237)). The transformed cells were then induced by growing them in galactose media, after which the cells were immediately plated on selective media lacking leucine (or histidine, in alternate rounds) to determine recombination efficiency. Typically ˜1-10% of induced cells were found to have undergone phenotype switching; this efficiency dropped by 2 to 3 orders of magnitude when the endonuclease, the subfragment with homology, or the galactose induction step was removed as a negative control (FIG. 10C), demonstrating that the double-strand break is essential to promote high frequency marker conversion. Literature precedent suggests the efficiency of cleavage and repair by homologous recombination could be increased to near 100% with optimization of induction conditions (Inbar et al., 1999, Molecular and Cellular Biology 19(6): 4134-4142). In the experiments described in this section, the efficiency of recombination was maintained even when the donor and acceptor subfragments shared only 40 bp of homology, which is short enough to easily be incorporated with PCR primers.

Recombinants were grown on selective media containing FOA (5-fluoroorotic acid, a counterselection for URA3) to cure cells of donor plasmids before proceeding to the next round of elongation. Cured recombinants were assayed phenotypically and genotypically to verify correct integration. Auxotrophies for histidine and leucine alternated with each round of elongation (FIG. 10D, columns). The newly integrated reporter (lacZ, gusA, or MET15) was functional in 75-100% of recombinants when ˜50 individual colonies from each round were assayed, and previously integrated markers were maintained (FIG. 10D, rows).

It was also demonstrated that integration occurred in the expected manner by analyzing the purified genomic DNA of 4-6 cured recombinants from each round via PCR and restriction digests (FIG. 10E).

### 7. EXAMPLE II

### E. coli Shuttle Vector

To demonstrate that reiterative recombination is not limited to the assembly of DNA only for yeast, we reconstructed a previously described, three-gene pathway for tetradehydrolycopene synthesis in E. coli (FIG. 11A)(Schmidt-Dannert et al., 2000, Nat Biotechnol 18(7): 750-3). After the pathway had been reconstructed by reiterative recombination in yeast, plasmid gap repair (Orrweaver et al., 1983, Proc. Natl. Acad. Sci. U.S.A. 80(14): 4417-4421) was used to move the contruct onto an E. coli shuttle vector. Though gap repair is an established technique for applications such as the retrieval of mutant alleles (Rothstein, 1991, Methods in Enzymology 194: 281-301.), recovery of the carotenoid pathway was inefficient, so a fourth round of elongation was used to add a gene for kanamycin resistance, giving a 5-kb construct. E. coli retransformed with the recovery vector were selected for kanamycin resistance, leading to identification of a plasmid with the intact construct. Colonies with this plasmid had the same colorimetric phenotype due to tetradehydrolycopene production as those with the previously reported plasmid (FIG. 11B) (Schmidt-Dannert et al., 2000, Nat Biotechnol 18(7): 750-3). The shuttle vector containing the recovered plasmid was 15 kb, near the size limit for pMB origins of replication, and poor stability in E. coli likely contributed to our difficulty moving the construct.

### 8. EXAMPLE III

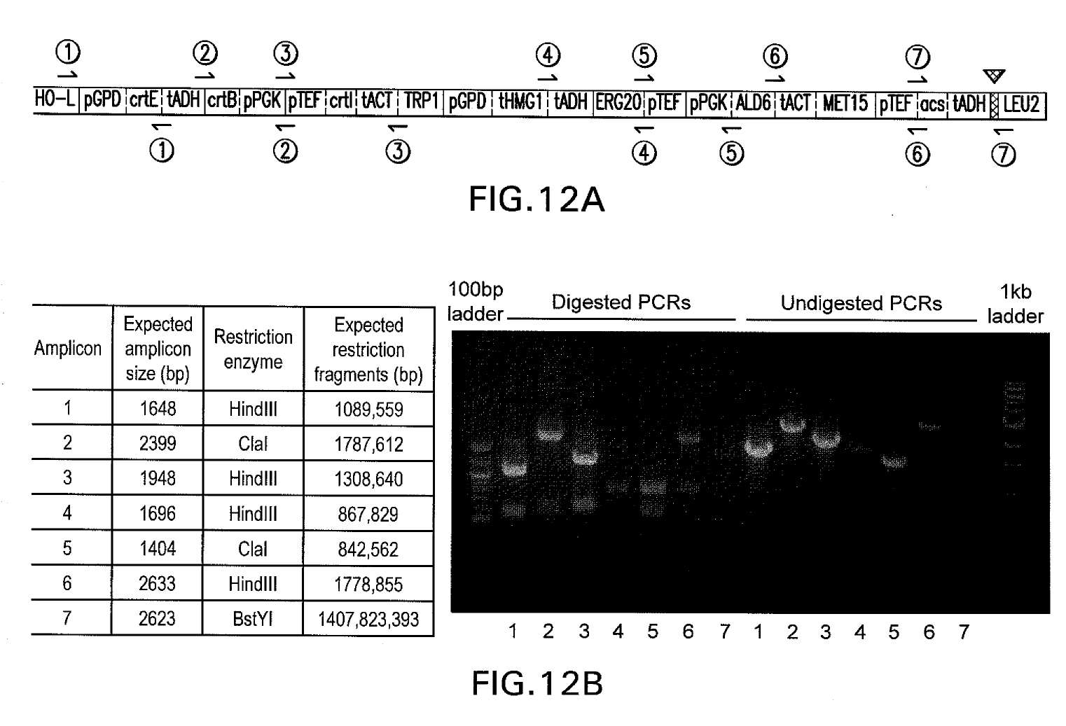

Reiterative recombination has been used to perform seven of eight rounds of elongation toward reconstructing the locus for the production of lycopene, demonstrating that reiterative recombination can be continued for at least seven rounds (as diagrammed in FIG. 12A) and used to build significantly larger constructs (18 kb) than in the working examples above. The methods set forth above were modified in that selection for TRP1 and MET15 markers was begun after they were integrated. The intermediate strains have exhibited the other expected phenotypes—alternating between HIS3 and LEU2 auxotrophies after each round, and turning orange once the three genes necessary for basal lycopene production (crtE, crtB, and crtI) were present. Assembly has been validated using PCR mapping and restriction digest of purified genomic DNA from cured recombinants (FIG. 12B). Excision of the repeated promoter and terminator sequences has not resulted in apparent problems, although it is not known how susceptible the strains are to the excision of repeats during normal growth or recombination absent selection for the intervening markers TRP1 and MET15.

Various publications are cited herein, the contents of which are hereby incorporated by reference in their entireties.

We claim:

1. A system for assembling DNA subfragments into a construct of interest, comprising:
(i) an acceptor module comprising, downstream to upstream (a) a first DNA subfragment that is to be assembled to form the construct; (b) an endonuclease cleavage site; and (c) a gene encoding a first selectable marker operably linked to (d) an active promoter or active promoter complex;
(ii) a first donor module comprising, downstream to upstream, (a) a second DNA subfragment, that is to be joined to the first DNA subfragment of (i) and that shares a region of homology with it; (b) an endonuclease cleavage site; (c) a gene encoding a second selectable marker which is not the same as the first selectable marker; and (d) a region upstream of the gene of (c) which is homologous to a region of the acceptor module such that recombination between the acceptor module and the first donor module in this region places the gene encoding the second selectable marker under transcriptional control of the active promoter or active promoter complex and inactivates the gene encoding the first selectable marker;
(iii) a second donor module comprising, downstream to upstream, (a) a third DNA subfragment, that is to be joined to the second DNA subfragment of (ii) and that shares a region of homology with it; (b) an endonuclease cleavage site; (c) a gene encoding the first selectable marker; and (d) a region upstream of the gene of (c) which is homologous to a region of the acceptor module such that recombination between the acceptor module and the first donor module in this region places the gene encoding the first selectable marker under transcriptional control of the active promoter or promoter complex and inactivates the gene encoding the second selectable marker; and
(iv) one or more nucleic acid encoding an endonuclease, a cleavage site of which is the site (b) present in the acceptor module and at least one donor module, operably linked to a promoter;
wherein the DNA construct is assembled by promoting endonuclease cleavage of the acceptor module, providing conditions that allow homologous recombination between the acceptor module and a donor module, and then selecting for a progeny acceptor module in which the marker has switched between the first selectable marker and the second selectable marker, indicative that homologous recombination has successfully occurred.

2. The system of claim 1, wherein the acceptor module is integrated into a host cell chromosome.

3. The system of claim 1, wherein the acceptor module is not integrated into a host cell chromosome.

4. The system of claim 1, wherein neither the first donor module nor the second donor module is integrated into a host cell chromosome.

5. The system of claim 1, wherein the first donor module is integrated into a host cell chromosome and the second donor module is integrated into a host cell chromosome.

6. The system of claim 1, wherein the endonuclease cleavage site of the acceptor module and at least one donor module is selected from the group consisting of the cleavage site of HO endonuclease, the cleavage site of SceI endonuclease, and the cleavage site of DmoI variant endonuclease.

7. The system of claim 1, wherein the endonuclease is selected from the group consisting of HO endonuclease, SceI endonuclease, and DmoI variant endonuclease.

8. A method for assembling a DNA construct of interest from a series of subfragments comprising
(i) providing overlapping subfragments of the construct,
(ii) providing a site-specific endonuclease which creates a double-strand break at a site between a subfragment or linked subfragments and an active promoter or promoter complex operably linked to a selection marker, thereby triggering homologous recombination,
(iii) selecting for a switch of the selection marker operably linked to said promoter or promoter complex from one to another alternative, which is indicative of homologous recombination; and
(iv) successively exchanging overlapping construct fragments, with each successive fragment extending the construct in the upstream direction so that the DNA construct is assembled.

9. A method for assembling a DNA construct of interest from a series of subfragments, comprising:
(i) providing an acceptor cell containing an acceptor module comprising, downstream to upstream (a) a first DNA subfragment that is to be assembled to form the construct; (b) an endonuclease cleavage site; and (c) a gene encoding a first selectable marker operably linked to (d) an active promoter or active promoter complex;
(ii) providing a first donor cell containing a first donor module comprising, downstream to upstream, (a) a second DNA subfragment, that is to be joined to the first DNA subfragment of (i) and that shares a region of homology with it; (b) an endonuclease cleavage site; (c) a gene encoding a second selectable marker which is not the same as the first selectable marker; and (d) a region upstream of the gene of (c) which is homologous to a region of the acceptor module such that recombination between the acceptor module and the first donor module in this region places the gene encoding the second selectable marker under transcriptional control of the active promoter or active promoter complex and inactivates the gene encoding the first selectable marker;
(iii) providing a second donor cell containing a second donor module comprising, downstream to upstream, (a) a third DNA subfragment, that is to be joined to the second DNA subfragment of (ii) and that shares a region of homology with it; (b) an endonuclease cleavage site; (c) a gene encoding the first selectable marker; and (d) a region upstream of the gene of (c) which is homologous to a region of the acceptor module such that recombination between the acceptor module and the first donor module in this region places the gene encoding the first selectable marker under transcriptional control of the active promoter or promoter complex and inactivates the gene encoding the second selectable marker;
(iv) allowing genetic exchange to occur between the acceptor cell and the first donor cell;
(v) providing an endonuclease that cleaves at cleavage site (b) in the acceptor module, thereby promoting homologous recombination;
(vi) selecting a progeny acceptor cell, resulting from steps (iv) and (v), that expresses the second selectable marker;
(vii) allowing genetic exchange to occur between the progeny acceptor cell and the second donor cell;
(viii) providing an endonuclease that cleaves at cleavage site (b) in the first donor module; and
(ix) selecting a progeny acceptor cell, resulting from steps (vii) and (viii), that expresses the first selectable marker;
to produce a construct whereby the first, second and third DNA subfragments are joined.

10. The method of claim 9, wherein after step (ix), the resulting progeny acceptor cell becomes the acceptor cell of step (i) and the method is repeated until assembly of the DNA construct is completed.

11. The method of claim 9, wherein the endonuclease cleavage site of the acceptor module and at least one donor module is selected from the group consisting of the cleavage site of HO endonuclease, the cleavage site of SceI endonuclease, and the cleavage site of DmoI variant endonuclease.

12. The method of claim 10, wherein the endonuclease cleavage site of the acceptor module and at least one donor module is selected from the group consisting of the cleavage site of HO endonuclease, the cleavage site of SceI endonuclease, and the cleavage site of DmoI variant endonuclease.

13. The method of claim 9, wherein the endonuclease is selected from the group consisting of HO endonuclease, SceI endonuclease, and DmoI variant endonuclease.

14. The method of claim 10, wherein the endonuclease is selected from the group consisting of HO endonuclease, SceI endonuclease, and DmoI variant endonuclease.

15. A kit for assembling a DNA construct of interest from a series of subfragments, comprising:
(i) an acceptor cassette comprising (a) a restriction site for inserting a DNA subfragment of the complex to be assembled; (b) an endonuclease cleavage site; and (c) a gene encoding a first selectable marker operably linked to (d) an active promoter or promoter complex; and
(ii) an odd donor cassette comprising (a) a restriction site for inserting a DNA subfragment of the complex to be assembled, (b) an endonuclease cleavage site; (c) a gene encoding a second selectable marker which differs from the gene for a first selectable marker in the acceptor module; and (d) a region upstream of the gene of (c) which is homologous to a region of the acceptor cassette.

16. The kit of claim 15, wherein the endonuclease cleavage site of the odd donor cassette is different from the endonuclease cleavage site of the acceptor cassette and these two sites are cleaved by different endonucleases.

17. The kit of claim 15, wherein the odd donor cassette is comprised in a nucleic acid that further comprises a nucleic acid encoding an endonuclease that cleaves at the endonuclease cleavage site of the acceptor cassette, said nucleic acid operably linked to a promoter.

18. The kit of claim 17, wherein, in the nucleic acid comprising the odd donor cassette, the promoter operably linked to the nucleic acid encoding the endonuclease is an inducible promoter.

19. The kit of claim 15, wherein the odd donor cassette is comprised in a nucleic acid that further comprises a nucleic acid encoding a third selectable marker different from the first and second selectable markers, said nucleic acid operably linked to a promoter.

20. The kit of claim 15, further comprising an even donor cassette comprising (i) a restriction site for inserting a DNA subfragment of the complex to be assembled, (ii) the same endonuclease cleavage site which is present in the acceptor cassette; (iii) a gene encoding the first selectable marker; and (iv) a region upstream of the gene of (iii) which is homologous to a region of the odd donor cassette or the acceptor cassette.

21. The kit of claim 20, wherein the endonuclease cleavage site of the even donor cassette is different from the endonuclease cleavage site of the odd donor cassette and these two sites are cleaved by different endonucleases.

22. The kit of claim 20, wherein the even donor cassette is comprised in a nucleic acid that further comprises a nucleic acid encoding an endonuclease that cleaves at the endonuclease cleavage site of the odd donor cassette, said nucleic acid operably linked to a promoter.

23. The kit of claim 22, wherein, in the nucleic acid comprising the even donor cassette, the promoter operably linked to the nucleic acid encoding the endonuclease is an inducible promoter.

24. The kit of claim 20, wherein the even donor cassette is comprised in a nucleic acid that further comprises a nucleic acid encoding a third selectable marker different from the first and second selectable markers, said nucleic acid operably linked to a promoter.

25. The kit of claim 15 or 20 which further comprises a nucleic acid encoding an endonuclease operably linked to an inducible promoter.

26. A method for combining a first DNA construct with a second DNA construct by convergent reiterative recombination by a method comprising:
(A) preparing the first DNA construct by a method comprising:
(Ai) providing a first acceptor cell containing a convergent acceptor module comprising, downstream to upstream (a) a first DNA subfragment that is to be assembled to form the construct; (b) an endonuclease cleavage site; and (c) a gene encoding a first selectable marker operably linked to (d) an active promoter or active promoter complex, wherein the active promoter or promoter complex is flanked by direct repeats;
(Aii) providing a first donor cell containing a first donor module comprising, downstream to upstream, (a) a second DNA subfragment, that is to be joined to the first DNA subfragment of (i) and that shares a region of homology with it; (b) an endonuclease cleavage site; (c) a gene encoding a second selectable marker which is not the same as the first selectable marker; and (d) a region upstream of the gene of (c) which is homologous to a region of the convergent acceptor module such that recombination between the convergent acceptor module and the first donor module in this region places the gene encoding the second selectable marker under transcriptional control of the active promoter or active promoter complex and inactivates the gene encoding the first selectable marker; and
(Aiii) allowing for genetic exchange between the first donor cell and the first acceptor cell;
(Aiv) selecting for expression of the second selectable marker; and then, after culturing under conditions that allow homologous recombination,
(Av) selecting for lack of expression of the second selectable marker (i.e., counterselecting), which indicates that the promoter element has been excised, such that the module contained by the a cell selected in this subparagraph has been transformed into a (third) donor module comprising the first DNA construct and said cell is a third donor cell;
(B) preparing the second DNA construct by a method comprising:
(Bi) providing a second acceptor cell containing a second acceptor module comprising, downstream to upstream (a) a third DNA subfragment that is to be assembled to form the construct; (b) an endonuclease cleavage site; and (c) a gene encoding a third selectable marker (which may or may not be the same as the first or second selectable markers) operably linked to (d) an active promoter or active promoter complex;
(Bii) providing a second donor cell containing a second donor module comprising, downstream to upstream, (a) a fourth DNA subfragment, that is to be joined to the third DNA subfragment of (i) and that shares a region of homology with it; (b) an endonuclease cleavage site; (c) a gene encoding a fourth selectable marker which is not the same as the third selectable marker; and (d) a region upstream of the gene of (c) which is homologous to a region of the acceptor module such that recombination between the acceptor module and the second donor module in this region places the gene encoding the fourth selectable marker under transcriptional control of the active promoter or active promoter complex and inactivates the gene encoding the third selectable marker; and
(Biii) allowing for genetic exchange between the second donor cell and the second acceptor cell;
(Biv) selecting for expression of the fourth selectable marker, wherein the module in a cell expressing the fourth selectable marker is a third acceptor module comprising the second DNA construct and said cell is a third acceptor cell; and
(C) allowing for genetic exchange between the third acceptor cell of (Biv) and the third donor cell of A(v); and
(D) selecting for the second selectable marker, wherein the module in a cell expressing the second selectable marker comprises the second DNA construct joined to the first DNA construct.

27. The method of claim 26 wherein the second acceptor module is a convergent acceptor module.

28. A shuttle acceptor module that may be used to assemble a DNA construct in a yeast, where a gene or genes of the assembled DNA construct are to be expressed in a bacterium, comprising downstream to upstream (a) a first DNA subfragment that is to be assembled to form the construct; (b) an endonuclease cleavage site; and (c) a gene encoding a first selectable marker operably linked to (d) an active promoter or active promoter complex, and (e) one or more element selected from the group consisting of an origin of replication utilized in the bacterium, a selection marker for the bacterium, and/or restriction endonuclease cleavage sites on either side of the gene or genes to be expressed.
